(19) United States
(12) Patent Application Publication
(10) Pub. No.: US 2026/0191912 A1
(43) Pub. Date: Jul. 9, 2026

(54) A NEW SITE FOR TRANSPLANTATION

(71) Applicant: HANGZHOU REPROGENIX BIOSCIENCE, INC., Hangzhou, Zhejiang (CN)

(72) Inventors: Yuanyuan DU, Hangzhou (CN); Zhen LIANG, Hangzhou (CN); Zhengjun LEI, Hangzhou (CN)

(21) Appl. No.: 18/868,497

(22) PCT Filed: May 25, 2023
(86) PCT No.: PCT/CN2023/096328

§ 371 Date: Nov. 22, 2024

(30) Foreign Application Priority Data
May 25, 2022 (WO) PCT/CN2022/095086

(51) Int. Cl. A61K 35/39; A61B 17/00; A61K 35/545; A61P 3/10

(52) U.S. Cl. CPC A61K 35/39; A61B 17/00; A61K 35/545; A61P 3/10; A61B 2017/00969

(57) ABSTRACT

Methods for transplanting cells or tissues, in particular islets, into a sub-rectus sheath site. Methods for treating diabetes by transplanting islets, particularly hPSC-islets, into a sub-rectus sheath position. The sub-rectus sheath position is a new site for transplantation.

### CROSS-REFERENCE

This application claims benefit of PCT International Application No. PCT/CN2022/095086 filed on May 25, 2022, which is incorporated herein by reference in its entirety.

### FIELD OF THE INVENTION

The present invention relates to the field of transplantation of cells, tissues or organs. Specifically, the present disclosure provides a method for transplanting cells or tissues into a sub-rectus sheath site. After transplantation, the cells or tissues are grafted under the rectus sheath, and preferably most of the cells or tissues are grafted within a space between the rectus sheath and the musculus rectus abdominis. The new transplantation site of the present invention is particularly suitable for heterotopic transplantation, e.g. of hormone-secreting cells or tissues. Also, the new transplantation site provides a desirable environment for transplants that are not fully differentiated or mature at the time of transplantation.

### BACKGROUND

Human pluripotent stem cells have shown great potential in cell replacement therapy for the treatment of diseases including diabetes. Our previous study in nonhuman primates had shown the feasibility of transplanting human pluripotent stem cell derived islets (hPSC-islets) to treat type I diabetes.

However, challenges remain in clinical application of hPSC-derived cells or tissues including hPSC-islets, one of which is the lack of a transplantation site effectively supporting functional maturation and long-term survival of transplants.

Take hPSC-islets as an example, intraportal infusion is most commonly used for clinical islet transplantation; however, it is far from satisfactory for reasons including the substantial loss of islets in the early transplantation stage, low revascularization of transplanted islets and progressive loss of graft function in the long term. To maintain a therapeutically effect level of functional islets, an excess amount of cells are needed for the transplantation so as to compensate the loss. However, such excess amount may lead to hypoglycemia. In addition, liver implantation results in the direct exposure of hPSC-islets to the high concentration of immunosuppressive drugs as well as the coagulation and complement system, which would further hinder their functional maturation and long-term maintenance. Previous experiences on mouse model show that sub-renal capsule is ideal for various cells and tissues transplantation, including both primary human islets and hPSC-islets (Du, Y. et al. Human pluripotent stem-cell-derived islets ameliorate diabetes in non-human primates. Nat Med 28, 272-282, doi: 10.1038/s41591-021-01645-7 (2022); Pagliuca, F. W. et al. Generation of functional human pancreatic beta cells in vitro. Cell 159, 428-439, doi: 10.1016/j. cell. 2014.09.040 (2014); Rezania, A. et al. Reversal of diabetes with insulin-producing cells derived in vitro from human pluripotent stem cells. Nat Biotechnol 32, 1121-1133, doi: 10.1038/nbt. 3033 (2014)). But this site is not suitable for clinical use due to the limited space for a high transplant mass and the invasive surgical procedure.

Much effort has been made to investigate an alternative site for islet transplantation on animal models. Successes were achieved in rodent models, but few transplantation sites could be transferred in large animal models.

A transplant site that can provide a permissive microenvironment for cell survival, growth and maintenance of the transplants, especially hPSC-derived transplants, is still needed in the art.

### SUMMARY OF THE INVENTION

The present inventors have discovered and that when transplanted under the anterior rectus sheath, hPSC-islets gradually matured and showed excellent graft function, which led to the overall improvement of glycemic control in diabetic non-human primates. Notably, C-peptide secretion dramatically increased and responded to meal challenge from 6 weeks post-transplantation (wpt), with the stimulation indices comparable to that of native islets. The average postprandial C-peptide level maintained at around 2.0 ng ml⁻¹ from 8 wpt, which was five-time higher than that of intraportal infusion. Accordingly, the average HbA1c decreased by over 44% at 12 wpt. Furthermore, the recipient macaques recovered quickly from cell transplantation, and showed lower risk of hypoglycemia in the first 24 hours. Collectively, the data demonstrate that the sub-anterior rectus sheath is an optimal transplantation site for hPSC-islets, which could be expanded to other derivatives of hPSC. This simple and noninvasive transplantation strategy combined with hPSC derivatives may introduce a new paradigm for cell replacement therapy, and thus complete the invention.

Accordingly, in a first aspect, the present application provides a method for transplanting one or more cells or tissue or to a subject in need thereof, comprising introducing the cells or tissues at a site under the rectus sheath (a sub-rectus sheath site) of the subject, preferably at a site between the rectus sheath and the musculus rectus abdominis. In some embodiments, the cells or tissues to be transplanted can be hPSC-derived cells or tissues. In some embodiments, the cells or tissues to be transplanted are those suitable for heterotopic transplantation, for example secretory cells, such as endocrine glands. In a particular embodiment, the cells or tissues to be transplanted are hPSC-derived islets. In another aspect, the method allows one or more of the transplanted cells or tissues to graft at a site under the rectus sheath after transplantation, preferably at a site between the rectus sheath and the musculus rectus abdominis.

In a second aspect, the present application provides a method for treating a disease or condition caused by or related to insufficiency of insulin in a subject in need thereof, comprising transplanting islets to the subject, wherein the islets are introduced at a site under the rectus sheath (a sub-rectus sheath site). In some embodiments, the islets are derived from hPSCs. In some embodiments, the disease is diabetes, or a complication thereof.

In a third aspect, the present application provides a method for reducing the requirement of exogenous insulin of a subject having diabetes, comprising transplanting islets to the subject, wherein the islets are introduced at a site under the rectus sheath (a sub-rectus sheath site). In some embodiments, the islets are derived from hPSCs.

In a fourth aspect, the present application provides use of islets in treating a disease or condition caused by or related to insufficiency of insulin, wherein the hPSC-derived islets are introduced at a site under the anterior rectus sheath (a sub-anterior rectus sheath site). In some embodiments, the disease is diabetes, or a complication thereof.

In a fifth aspect, the present application provides a device for injecting islets, e.g. hPSC-derived islets to a site under the rectus sheath (a sub rectus sheath site), preferably into the space between anterior rectus sheath and musculus rectus abdominis. In some embodiments, the device comprises a needle and a cartridge containing a suspension of hPSC-derived islets. In a preferred embodiment, the device comprises a puncture needle. In a more preferred embodiment, the puncture needle having single or multiple needle tracks, e.g. 1, 2, 3, 4, 5, 6, 7, 8, 9, 10, or more needle tracks.

The new transplant site of the present invention at least provides following advantages.
(1) Sub-rectus sheath transplantation of hPSC-islets have achieved desirable therapeutic effects which show functionality comparable to that of primary human islets of a healthy adult.
(2) The musculus rectus abdominis has abundant blood vessels, and is enclosed by rectus sheath, which provides the grafts with a desirable microenvironment for survival, growth and maintenance after sub-rectus sheath transplantation.
(3) As an extraperitoneal site, sub-rectus sheath transplantation of hPSC-islets can be completed by a simple and noninvasive manner, such as by infusion at bedside. Such convenient procedure allows for repeated infusions and quick recovery after the transplant procedure.
(4) Sub-rectus sheath transplantation allows for removal of the transplants in any case where the recipient no longer benefits from the transplanted tissues. For example, as the long-term safety of hPSC-derived tissues is still under investigation, it may be desirable to leave the window open for removing the hPSC-islets.
(5) Intraportal transplantation of islets triggers instant blood-mediated inflammatory reaction (IBMIR) and activates the coagulation. Therefore, application of anticoagulants is continuously needed during intraportal transplantation. Inappropriate control of coagulation may result in hazardous or even lethal hypercoagulability (thrombosis) or hypocoagulability (bleeding). IBMIR is reported to lead to dramatic β-cell death and in turn a drop of blood glucose. By transplanting at the new site of the present invention, no hypoglycemic due to β-cell death is observed, suggesting the problem due to IBMIR has been solved.
(6) The new transplant site under rectus sheath can also be used for transplantations of a wide variety of cells or tissues. Especially preferred transplants are hPSC-derived cells or tissues, e.g. hPSC-islets.

### BRIEF DESCRIPTION OF THE DRAWINGS

FIG. 1a-c | Schematic of hPSC-islet transplantation strategy. a, hPSC-islets were transplanted into three extraperitoneal sites for comparison. b, Under the guidance of ultrasound imaging, hPSC-islets were transplanted into the sub-anterior sheath using a puncture needle. c, hPSC-islet was delivered over eight injections in four parallel needle tracks on each side of the abdominis rectus.

FIG. 2a-f | Characterization of hPSC-islets used for transplantation. a, Representative bright field image of hPSC-islets. Scale bar, 200 μm. b, Representative flow cytometry data depicting islet hormone and transcription factor expression in hPSC-islets. c, Representative immunofluorescence staining of islet hormones in sectioned hPSC-islets. Scale bar, 25 μm. d, Representative immunostaining of key β cell transcription factors and C-peptide in sectioned hPSC-islets. Scale bar, 50 μm. e, C-peptide secretion of hPSC-islets (n=3) in static glucose stimulation assays under low glucose (2.8 mM), high glucose (16.7 mM) and depolarization by 30 mM KCl. Glucose stimulation indices are indicated above the bars. f, qRT-PCR analysis of key pancreatic islet genes in hPSC-islets (n=4) and human islets (n=3).

FIG. 3a-c | Immunostaining of hPSC-islet grafts in three extraperitoneal transplant sites in Monkey 1 at 1-week post transplantation. a, Representative immunohistological staining of human cell marker Stem121. Scale bars, 400 μm; for the enlarged view (indicated with green border), 50 μm. (b-c), Representative immunofluorescence staining of C-peptide and β cell specific transcription factor PDX1 (b) or NKX2.2 (c) in human grafts. Scale bar, 50 μm.

FIG. 4a-d | Evaluation of hPSC-islet grafts in three extraperitoneal transplant sites in Monkey 1 at 1 week post transplantation. a, Quantitative assessment of the volume of hPSC-islet grafts in three indicated sites. b, The proportion of C-peptide expressing cells in the human grafts at three indicated graft sites (n=12). c, Representative immunofluorescence staining of islet hormones in hPSC-islet grafts. d, Representative immunofluorescence staining of C-peptide and β cell specific transcription factor NKX6.1 in human grafts. Scale bar, 50 μm. Data presented as mean±SEM.

FIG. 5a-e | Evaluation of hPSC-islet grafts in Monkey 2 at 4 weeks post transplantation. a, Representative immunohistological staining image of human cell marker Stem121 in three indicated graft sites. Scale bars, 400 μm; for the enlarged view (indicated with green border), 50 μm. (b-c), Representative immunofluorescenc e staining of islet hormones, pancreatic transcription factors and UCN3 in sub-anterior rectus sheath grafts. Scale bar, 50 μm. d, Representative immunohistological staining of endothelial marker CD31 in sub-anterior rectus sheath grafts. Scale bars, 100 μm; for the enlarged view (indicated with green border), 25 μm. e, Representative immunohistological staining of T cell marker CD3, B cell marker CD20 and macrophage marker CD68 in sub-anterior rectus sheath grafts. Scale bars, 100 μm; for the enlarged view (indicated with green border), 25 μm.

FIG. 6a-e | Evaluation of hPSC-islet grafts in Monkey 3 at 4 weeks post transplantation. a, Representative immunohistological staining of human cell marker Stem121 in sub-anterior rectus sheath grafts. Scale bars, 400 μm; for the enlarged view (indicated with green border), 50 μm. (b-c), Representative immunofluorescence staining of islet hormones, pancreatic transcription factors and UCN3 in sub-anterior rectus sheath grafts. Scale bar, 50 μm. d, Representative immunohistological staining of endothelial marker CD31 in sub-anterior rectus sheath grafts. Scale bars, 100 μm; for the enlarged view (indicated with green border), 25 μm. e, Representative immunohistological staining of T cell marker CD3, B cell marker CD20 and macrophage marker CD68 in sub-anterior rectus sheath grafts. Scale bars, 100 μm; for the enlarged view (indicated with green border), 25 μm.

FIG. 7a-1 | hPSC-islet transplantation under the anterior rectus sheath resulted in improvement of glycemic control in immunosuppressed diabetic rhesus macaques. a-d, Continuous tracking of fasting, pre- and post-prandial blood glucose pre- and post-hPSC-islet transplantation under the anterior rectus sheath of STZ-treated monkeys. Each data point represents a weekly value based on averaged blood glucose (n=7 independent values) except −5 wpt of Monkey 5 (n=5 independent values), −5 wpt of Monkey 6 (n=6 independent values) and −5 wpt of Monkey 7 (n=6 independent values). e-h, Continuous tracking of glycated hemoglobin (HbA1c) levels in the diabetic macaque recipients pre-STZ treatment (grey), pre-hPSC-islet transplantation (0 wpt, brown) and post-hPSC-islet transplantation (black). i-1, Continuous tracking of the exogenous insulin requirements of the diabetic macaque recipients pre- and post-hPSC-islet transplantation. Each data point represents a weekly value based on averaged daily exogenous insulin doses (n=7 independent dose values). Exogenous insulin doses at the last week before transplantation and at 12-wpt are indicated in brown. Data presented as mean±SEM.

FIG. 8a-h | Evaluation of hPSC-islet grafts in Monkey 3 at 4-week post transplantation. a-d, Daily fasting blood glucose levels of diabetic macaque recipients before STZ treatment (grey), before (brown) and after (black) sub-anterior rectus sheath transplantation of hPSC-islets (infusion procedure conducted at day 0). e-h, C-peptide secretion in response to glucose potentiated arginine (Arg) stimulation, conducted at 12 wpt (n=3, technical replicates) and pre-transplantation.

FIG. 9a-1 | C-peptide secretion of diabetic rhesus macaques with hPSC-islets transplanted under the anterior rectus sheath. a-d, Detection of fasting (grey) and post-prandial (red) C-peptide secretion in diabetic macaque recipients pre-STZ treatment, pre-hPSC-islet transplantation (0 wpt) and post hPSC-islet transplantation (n=3 technical replicates). Stimulation indices are indicated above the bars. e-1, Changes in blood glucose levels (e-h) and C-peptide levels (i-1) upon intravenous glucose tolerance test (IVGTT) administered to the diabetic macaque recipients pre-transplant (Pre-Tx) and at 4, 8 and 12 wpt (technical replicates; n=2 (e-h); n=3 (i-1)).

FIG. 10a-d | Gross anatomy, histological and immunological analysis of native pancreas of STZ-treated recipient monkeys. a, Gross anatomy and H&E staining of pancreas sections of recipient monkeys, with islet structures outlined in green. Scale bar, 100 μm. b, C-peptide staining of pancreas sections of STZ-treated recipient monkeys. Scale bars, 200 μm; for the enlarged view (indicated with green border), 50 μm. c-e, Representative immunofluorescence staining of CHGA (c), islet hormones (d) and CK19 and Proinsulin (e) in pancreas sections of STZ-treated recipient Monkeys. Scale bar, 50 μm.

FIG. 11a-g | Characterization of sub-anterior rectus sheath hPSC-islet grafts in Monkey 4 at 13 wpt. a, Representative immunohistological staining of human cell marker Stem121 in sub-anterior rectus sheath grafts. Scale bars, 400 μm; for the enlarged view (indicated with green border), 100 μm. (b-c), Representative immunofluorescence staining of islet hormones, pancreatic transcription factors and UCN3 in sub-anterior rectus sheath grafts. Scale bar, 50 μm. d, The proportion of C-peptide expressing cells in human grafts (n=10). e, The proportion of C-peptide expression in PDX1 or NKX6.1 positive cells in the hPSC-islet grafts (n=4). f, The proportion of MAFA expression in C-peptide positive cells in the hPSC-islet grafts (n=4). g, Representative immunohistological staining of endothelial marker CD31 in sub-anterior rectus sheath grafts. Scale bars, 100 μm; for the enlarged view (indicated with green border), 25 μm.

FIG. 11a-g | Characterization of sub-anterior rectus sheath hPSC-islet graft in Monkeys 5-7 at 13 wpt. (a-f), Representative immunofluorescence staining of islet hormones, pancreatic transcription factors and UCN3 in sub-anterior rectus sheath grafts. Scale bar, 50 μm. g, Representative immunohistological staining of endothelial marker CD31 in sub-anterior rectus sheath grafts. Scale bars, 100 μm; for the enlarged view (indicated with green border), 25 μm.

FIG. 12a-1 | Ultrasound examination and postmortem examination of major organs in transplanted diabetic macaques. Gross anatomy (a, d, g and j), H&E staining (b, e h and k) and ultrasound examination (c, f, i and 1) of major organs of Monkey 4-7. Scale bar, 200 μm.

### DETAILED DESCRIPTION OF THE INVENTION

Unless specifically defined elsewhere in this document, all the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

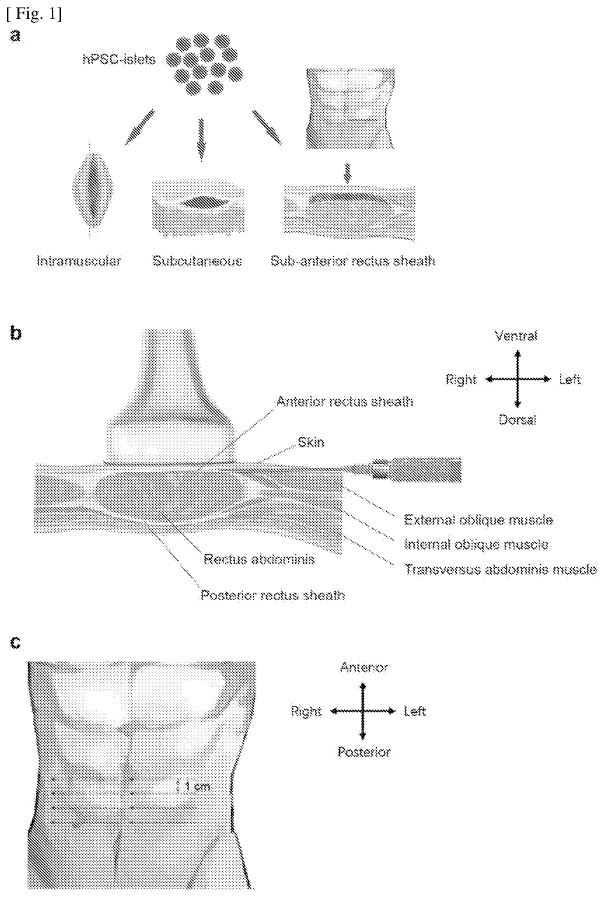

By “sub-rectus sheath transplantation”, it refers to introduce the transplants or grafts into a position beneath and within the rectus sheath, preferably into the space between the rectus sheath and musculus rectus abdominis. By “sub-anterior rectus sheath transplantation”, it specifically refers to administering the transplants or grafts into a position under the front side of the rectus sheath, preferably into the space between the anterior rectus sheath and the front side of the rectus abdominis, and reaching a position under the anterior rectus sheath, as shown in FIGS. 1a and 1b.

The term “rectus abdominis”, “abdominis rectus”, “musculus rectus abdominis” or “abdominal muscle” can be used interchangeably in the context of the present invention, which refer to the two parallel flat muscles on either side of the linea alba and extending along the whole length of front of the abdomen.

The term “subject” refers to an animal, preferably a mammal, e.g., a non-human primate or preferably human. In some cases, the subject is the “recipient” of the transplants.

The term “secretory cell” refers to any cell having a function of generating a substance and secreting the substance to the space out of the cell.

The term “islet” herein refers to cell clusters containing insulin producing cells, which are capable of regulating blood glucose level.

The term “islet equivalent” or “IEQ” is a standardized measure to describe the islet mass to be transplanted. Reference can be made to e.g. Lembert N, et al., for the determination of IEQ (Lembert N, et al. Areal density measurement is a convenient method for the determination of porcine islet equivalents without counting and sizing individual islets. Cell Transplant. 2003; 12 (1): 33-41)

The term “pluripotent stem cells (PSCs)” herein refers to undifferentiated cells defined by their ability, at the single cell level, to both self-renew and differentiate. Stem cells may produce progeny cells, including self-renewing progenitors, non-renewing progenitors, and terminally differentiated cells. Stem cells may be characterized by their ability to differentiate into functional cells of various cell lineages from multiple germ layers (endoderm, mesoderm, and ectoderm). The abbreviation “hPSC” refers to human pluripotent stem cells. Preferably, hPSCs in the present method are induced pluripotent stem cells (iPSCs), or embryonic stem cells (ESCs) obtained from embryos that have not been developed in vivo and are within 14 days after fertilization.

The term “hPSC-derived” cells or tissues refer to those cells or tissues generated or differentiated from human pluripotent stem cells.

The term “hPSC-islets” refers to islets including insulin producing cells, which were derived from human pluripotent stem cells.

### Transplantation Site

The merit of the present application lies in the discovery of the new transplant site which locates beneath and within the sheath of rectus abdominis, preferably within or close to the space between the rectus sheath and rectus abdominis. By “transplantation site” in the present invention, it refers to the site where the one or more cells or tissues are delivered and introduced. By the expression “cells or tissues graft at a site”, it means that the transplanted cells or tissues stay and grow at the site after transplantation. In some cases, the site where the cells or tissues graft is not perfectly identical or 100% overlap with the transplantation site, since a part of the transplanted cells may migrate to some extents.

To facilitate the understanding, reference is made to FIG. 1a-c to describe the new transplantation site of the present application. As shown by FIGS. 1a and 1c, the transplants or grafts are delivered into a place between the sheath of rectus abdominis and the surface of rectus abdominis. However, due to the accuracy of practitioner's operation, the precise injection site may vary and sometimes can be slightly deeper into the superficial musculus rectus abdominis. In another embodiment, the transplants or grafts are delivered into the musculus rectus abdominis, preferably at a position close to the rectus sheath.

As shown in FIG. 1a, the rightmost two pictures illustrate a cross-sectional view of the human abdomen, showing an exemplary position where the transplants or grafts can be introduced in the present invention. The transplants are delivered to a position under the thin layer of rectus sheath (specifically the anterior rectus sheath in FIG. 1a) and in front of the rectus abdominis. After delivery, the transplants may spread along the rectus abdominis and form a layer of transplants under the rectus sheath. It should be understood that the needle can penetrate through any part of the skin, at any angle, to any depth, as long as it can reach the desired transplantation site.

The transplant site of the present application can be easily reached by routine administering means, such as by injection with a device comprising a needle. Therefore, cells or tissues to be transplanted can be delivered by a less invasive method such as injection or infusion. The transplant site of the present application also allows removal of the grafts in case of any unwanted side effect occurs after transplantation, or the transplants are not needed any more.

After the transplants or grafts are delivered into the desired site, most of the transplanted cells may stay in place, while some of the transplanted cells may also migrate within the sheath of rectus abdominis to a place away from the original transplant site. As long as the cells derived from the transplants or grafts remain in the sheath of rectus abdominis, it is within the scope of the present application.

The transplant site of the present application also provides an optimal environment for the maintenance of the transplants or grafts. Within the realm surrounded by the rectus sheath, a sufficient space can be provided for the transplants or grafts to grow, and abundant vascular networks serve the transplants or grafts with sufficient oxygen and nutrients to support their survival, growth and maintenance. In some cases, vascular infiltration into the transplants or grafts can be observed several weeks after transplantation.

The transplant site of the present application is especially suitable for “pre-mature” transplants or grafts which may need a maturation process in vivo after transplantation. For example, the transplants may comprise a certain portion of cells that are not fully developed or differentiated. Such a transplantation environment permissive to functional maturation is particularly important for cells or tissues derived from undifferentiated cells or cells that are not fully differentiated, especially stem cells, endoderm stem cell lines, mesenchymal stem cells, progenitor cells or precursor cells. In some embodiments, the transplants or grafts are cells or tissues derived from undifferentiated cells or cells that are not fully differentiated, e.g. those differentiated from hPSCs, which are referred as hPSC-cells or hPSC-tissues in the context of the present application. For example, cells or tissues derived from human pluripotent stem cells (hPSCs) can be those derived from embryonic stem cells, or induced pluripotent stem cells (iPSCs). In a specific embodiment, for transplantation of hPSC-derived islets, the post-transplantation maturation allows the improvement of secretion insulin capacity and sensitivity to glucose challenge of hPSC-islets, which is critical to the therapeutic effects of hPSC-islets.

### Transplantation Process

The present invention does not intend to limit the means to introduce the transplants into a site under the rectus sheath. The means or device used for conducting the transplantation may depend on the type of transplants, amount of transplants, and the like.

In some embodiments, the transplantation is conducted by injection. FIG. 1b exemplifies the sub-rectus sheath injection of transplants. As shown in FIG. 1b, a puncture need is manipulated to enter the body, first penetrate through skin and then the rectus sheath, and reach a position beneath the rectus sheath, preferably between the surface of the rectus abdominis and the (anterior) rectus sheath. Then, the needle is allowed to penetrate deeper while keeping to move along the space between the (anterior) rectus sheath and the rectus abdominis muscle. After the needle is in a desired place, injection of cells is started. While injecting the cells, the needle is slowly withdrawn along the needle track so as to leave the injected cells along the track which is between the rectus sheath and the rectus abdominis muscle. The present invention does not require all the transplants are injected or seeded within the space between the rectus sheath and the rectus abdominis muscle. It would be understood that a part of the transplanted cells may be seeded into the rectus abdominis muscle. As long as part of the transplants are within the rectus sheath, it is within the scope of the present invention.

The transplantation, e.g. by injection, can be conducted on either side, left or right, of the body, i.e. under the rectus sheath surrounding the left or right rectus abdominis, or conducted on both sides.

The transplantation, e.g. by injection, can be conducted by an injector such as a syringe. In case of injection, the injecting device can comprise a needle, such as a puncture needle, or any suitable needle known in the art. The needle should be configured to be sharp and rigid enough to penetrate skin and rectus sheath, and to be long enough to reach the desired position. The length of the needle can be any length between about 1-25 cm. The size of the needle can be selected depending on multiple factors, including the type, amount and formulation of transplants, as well as the needle insertion site.

For example, the needle can have an inner diameter of about 0.15 mm to 3 mm, e.g. 0.15 mm, 0.2 mm, 0.25 mm, 0.3 mm, 0.35 mm, 0.4 mm, 0.45 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm. In case of injecting islets, the needle can have an inner diameter of about 0.25 mm to 3 mm. One skilled in the art would be able to determine the gauge of the needle accordingly. For example, the needle can be any size between 12 gauge and 30 gauge. For example, in case of injecting islets, the needle can be any size between 12 gauge and 26 gauge.

For injection, the cells to be transplanted can be provided as a suspension, e.g. a suspension of cells in saline. When the total volume of the cell suspension is large, the injection can be conducted by a device with multiple needle tracks, e.g. 2, 3, 4, 5, 6, 7, 8, 9, 10 or more needle tracks, with each track having a portion of the total volume of the cell suspension.

The number of cells or tissues to be transplanted into a sub-rectus sheath position can range from 1 to about 1×10¹¹ cells in total. For example, the cells or tissues can comprise about 1 cell, 10 cells, 1× 10² cells, 1× 10³ cells, 1× 10⁴ cells, 1× 10⁵ cells, 1× 10⁶ cells, 1× 10⁷ cells, 1× 10⁸ cells, 1× 10⁹ cells, 1×10¹⁰ cells, or 1× 10¹¹ cells. For example, the cells or tissues to be transplanted and contained in the cartridge of an injector may comprise about 10 cells, 1× 10² cells, 1×10³ cells, 1×10⁴ cells, 1× 10⁵ cells, 1× 10⁶ cells, 1× 10⁷ cells, 1× 10⁸ cells, 1× 10⁹ cells, 1×10¹⁰ cells, or 1× 10¹¹ cells. In some embodiments, at least about 50%, 55%, 60%, 65%, 70%, 75%, 80%, 85%, 90%, 95%, or about 100% of the cells to be transplanted are viable cells.

For example, in an example of transplanting islets, the total amount of islets can range from 100 to 200,000 IEQ per kg body weight of the recipient, preferably 500 to 150,000 IEQ per kg body weight of the recipient, more preferably 5,000 to 80,000 IEQ per kg body weight of the recipient, or even more preferably 10,000 to 50,000 IEQ per kg body weight of the recipient.

To facilitate accurate deposition of the grafts, imaging technology such as ultrasound can be used to guide the transplantation. In one embodiment, ultrasound imaging is used to facilitate the transplantation.

### Type of Transplants

The new transplant site of the present application is suitable for the transplantation of a wide variety of transplants or grafts. The transplants of the present invention are preferably cells, tissues or organoids.

In the broadest scope of the present application, it is not intended to limit the type or source of transplants. The transplants can be cells, tissues or organoids provided by an individual, e.g. a donor, and/or prepared by in vitro method including induction, differentiation, maturation or the like.

In some embodiments, the transplants are delivered to a subject with a therapeutic purpose. In this case, the transplants can be cells or tissues with a desired function.

In some embodiments, the transplants are delivered to a subject with a non-therapeutic purpose. For example, deleterious transplants can be placed into an animal to establish disease model. In an example of this embodiment, the transplants can be cells or tissues derived from tumor.

The present application is particularly suitable for heterotopic transplantation. In this case, the transplants are ectopic transplants. The success of heterotopic transplantation relies on the ability of the ectopic transplants or grafts to survive and function properly at the new site, which is different from the place where they can be naturally found. In some embodiments, the transplants comprises or consists of tissues or cells of a secretory gland, such as endocrine glands, e.g. thyroid gland, parathyroid gland, hypothalamus, pituitary, adclarenal, pineal body. In some embodiments, the transplants or grafts comprise or consists of islets, pancreatic cells, hepatocytes, kidney cells, thymic cells, lung cells.

In some embodiments, the transplants or grafts of the present invention are autologous, allogenic, xenogeneic or syngeneic to the subject or recipient.

The transplants or grafts of the present invention may be derived from any species of animals, preferably mammals, more preferably primates, most preferably human.

The transplants of the present invention can comprise pre-mature cells or tissues not fully differentiated or developed. For example, when the transplants are prepared from pluripotent, multipotent cells, progenitor cells or precursor cells, the transplants may comprise a certain portion of such “immature” cells. In one embodiment, the transplants can be cells derived from an embryo, e.g. embryonic stem cells or cells derived from embryonic stem cells (ESCs), e.g. human embryonic stem cells. In one embodiment, the human embryonic stem cells are not undergoing in vivo development and are within 14 days after fertilization. In some embodiments, the transplants comprise fully differentiated, developed or immature cells which are allowed to undergo differentiation and/or maturation in the recipient after the transplantation.

In some embodiments, the transplants can be obtained by differentiation of pluripotent cells, e.g. ESCs or induced pluripotent stem cells (iPSCs), or multipotent cell, e.g. precursor cells or progenitor cells. Examples of precursor cells include endoderm progenitor cells, such as endoderm progenitor cells with the potential to differentiate into pancreas or liver.

In some specific embodiments, the transplants are islets. In the context of the present application, the term “islet” is understood in a broad sense to include any cells or cell aggregates that secrete insulin. The islets of the present application include islet organoids. The islets can be taken from an individual, e.g. a donor, including an allogenic donor, an autologous donor, or an xenogeneic donor. The islets can be derived from stem cells, including ESCs and iPSCs, or islet protenitor cells. The islets may include healthy islet cells or dysfunctional islet cells. In one specific embodiment, the islets are hPSC-islets which are differentiated from human iPSCs. The differentiation from stem cells to islets can be completed in vitro before the transplantation. Alternatively, the stem cells are subjected to differentiation in vitro into pre-mature islets, followed by maturation in vivo after transplantation.

The cells to be transplanted can be prepared into cell suspensions. In some embodiments, the cells can be dissociated by enzymes before transplantation. The enzyme for dissociating cells can include but not limited to Accutase, TrypLE, Versene, CTS™ TrypLE, and CTS™ Versene.

### Therapeutic Uses

The new transplant site of the present application has great potential in therapeutic uses, as it allows a less invasive procedure and a supportive environment for grafts. Depending on the type of transplants, the method of the present application can treat various diseases and conditions. The present methods may have potential uses in the transplantation of a wide range of tissue types, with a purpose of supplementing or reconstituting organ function. Particularly suitable organ tissues to be transplanted by the present method may include endocrine gland organ tissues, such as adrenal gland. Also interested are those organs for which orthotopic transplantation is not a must. For example, the present method can be used for heterotopic transplantation.

For example, the present method is particularly suitable for heterotopic transplantation of secretory cells producing hormones. In this cases, the present method can be used to treat disease or condition related to or caused by absence or insufficiency of a hormone.

In specific embodiments, the present method comprises sub-rectus sheath transplantation of islets, e.g. hPSC-derived islets, so as to treat conditions due to absence or insufficiency of an insulin, including various types of diabetes which can be affected by genetic, metabolic, environmental, and/or immune factors. The conditions may include hyperglycemia, type 1 diabetes (TID), type 2 diabetes (T2D), T1D-or T2D-associated complications, and other rare types of diabetes.

The sub-rectus sheath transplantation of islets may lead to increased secretion of C-peptide. The sub-rectus sheath transplantation of islets can improve long-term and short-term glycemic control. The glycemic control can be measured by HbA1c, fasting blood glucose, postprandial blood glucose, fluctuation of blood glucose. Subjects can benefit from the transplantation of the present invention as requirement for exogenous insulin can be reduced or eliminated.

In case of transplanting islets, the transplantation of the present invention allows desirable maintenance and maturation of the transplants. In some embodiments, the grafted islets are positive for mature β cell markers, such as MAFA and UCN3, after about 2 weeks, 3 weeks or 4 weeks after transplantation.

In one embodiment, the present method comprises sub-rectus sheath transplantation of hepatocytes.

The sub-rectus sheath transplantation of the present application can be used to replace current transplantation into portal vein.

### EXAMPLES

For a more complete understanding and application of the present invention, the present invention will be described in detail below with reference to the examples and the drawings. Those skilled in the art would understand that the following examples are only intended to illustrate the present invention without any intention to limit the scope of the present invention. The scope of the present invention should be defined by the appended claims.

### Methods

### Cell Culture

One chemically induced pluripotent human stem cell line hPSC-8 # was used in this study. hPSC-8 # cells were induced from human human adult adipose-derived cells using a chemical reprogramming strategy without gene transfer (Guan, J. et al. Chemical reprogramming of human somatic cells to pluripotent stem cells. Nature 605, 325-331, doi: 10.1038/s41586-022-04593-5 (2022)). hPSCs were cultured in mTeSR1 (STEMCELL Technologies, 85850) on a 1:40 diluted Matrigel-coated (BD BioSciences, 356231) six-well plate (Corning, 353046) or 500 cm² Square TC-treated Culture Dish (Corning, 431110) at 5% CO₂, 37° C. Medium was changed daily and cells were passaged every 5-6 d with ReleSR (STEMCELL Technologies, 05872) at a 1:10-1:15 split ratio. Meanwhile, cells were verified to be mycoplasma-free by using a MycoSEQ Mycoplasma Detection Kit (Thermo Fisher Scientific, 4460626).

Differentiation protocol to generate hPSC-islets.

hPSCs were differentiated with a six-stage protocol as previously described by the present inventors (Du, Y. et al. Human pluripotent stem-cell-derived islets ameliorate diabetes in non-human primates. Nat Med 28, 272-282, doi: 10.1038/s41591-021-01645-7 (2022)). In brief, hPSCs were dispersed into single cells with Accutase (EMD Millipore, SCR005) and seeded at ~1.35×10⁵ cells per cm² on Matrigel-coated Nunc™ EasyFill™-2 Cell Factory™ systems (Thermo Fisher Scientific, 169171) in mTESR1 supplemented with 10 μM Y27632. Differentiation was started 24 h after seeding by replacing the medium with a protocol-appropriate medium supplemented with small molecule or cytokines (Table 1 and Table 2). At the end of Stage 3, the cells were dispersed with Accutase. After rinsing with DMEM-basic medium, the cells were seeded at 5×10⁶ cells/well in six-well AggreWell Microwell Plates (STEMCELL Technologies, 27940) in Stage 4 medium supplemented with 10 μM Y27632 and spun down at 300 g for 5 min. After incubation in 5% CO₂ at 37° C. for 24 h, the clusters were transferred into an ultra-low attachment six-well plate (Beaver Bio, 40406) with Stage 4 medium. Suspended aggregates were cultured in an incubator shaker (INFORS HT, Multitron) at a rotation rate of 90 r.p.m., at 37° C., 5% CO₂ and 85% humidity. Detailed information for small molecules, cytokines, culture medium and supplements is listed in Table 3.

TABLE 1
Base media formulations

| Media | Ingredients | Media | Ingredients |
|---|---|---|---|
| M1 | MCDB131 | M2 | MCDB131 |
| | Glucose 4.5 mM | | Glucose 4.5 mM |
| | GlutaMAX 1% | | GlutaMAX 1% |
| | B27 1% | | BSA 0.5% |
| | Pen/Strep 1% | | Pen/Strep 1% |
| M3 | DMEM-Basic | M4 | DMEM-Basic |
| | B27 1% | | GlutaMAX 1% |
| | Pen/Strep 1% | | B27 1% |
| | | | Pen/Strep 1% |

TABLE 2
Specification of differentiation protocols

| | Day | | | | |
|---|---|---|---|---|---|
| Stage | number | | Media | Factors | |
| Stage | 1 | Day | M1 | Activin A 100 ng/mL | PI103 50 nM |
| 1 | | | | Vitamin C 0.25 mM | Y27632 10 μM |
| | | | | Chir99021 6 μM | |
| Stage | 3 | Days | M1 | Activin A 50 ng/mL | |
| 1 | | | | Vitamin C 0.25 mM | |
| Stage | 2 | Days | M2 | KGF 50 ng/mL | SB431542 5 μM |
| 2 | | | | Vitamin C 0.25 mM | Wnt-C59 100 nM |
| Stage | 4 | Days | M3 | Retinoic acid 2 μM | Sant1 0.25 μM |
| 3 | | | | LDN193189 0.1 μM | Wnt-C59 100 nM |
| Stage | 5-6 | Days | M4 | EGF 100 ng/mL | Sant1 0.25 μM |
| 4 | | | | TPB 0.2 μM | Vitamin C 0.25 mM |
| | | | | Nicotinamide 10 mM | |
| Stage | 6 | Days | M4 | ALK5 inhibitor II 10 | Xxi 0.1 μM |
| 5 | | | | μM | |
| | | | | LDN193189 0.3 μM | Wnt-C59 100 nM |
| | | | | T3 1 μM | Y27632 10 μM |
| | | | | ISX9 10 μM | Vitamin C 0.25 mM |
| | | | | Heparin 10 μg/mL | |
| Stage | 2-4 | Days | M3 | ALK5 inhibitor II 10 | Heparin 10 μg/mL |
| 6 | | | | μM | |
| | | | | R428 0.5 μM | Zinc sulfate 10 μM |
| | | | | T3 1 μM | Nac 2 mM |
| | | | | Forskolin 10 μM | Vitamin C 0.25 mM |

TABLE 3
Small molecules, cytokines, basal medium and supplements
used in the differentiation protocol.

| Name | Source | Cat# |
|---|---|---|
| Activin A | Stemimmune LLC | HST-A-1000 |
| KGF | Stemimmune LLC | HST-F7-1000 |
| EGF | Peprotech | AF-100-15 |
| Y27632 | Selleck | S1049 |
| Chir99021 | Selleck | S1263 |
| PI103 | Selleck | S1038 |
| Vitamin C (Vc) | Sigma | 49752 |
| SB431542 | Selleck | S1067 |
| Wnt-C59 | Selleck | S7037 |
| LDN193189 | Selleck | S7507 |
| Retinoic acid (RA) | Sigma | R2625 |
| Sant1 | Selleck | S7092 |
| Nicotinamide | Sigma | N0636 |
| TPB | Adooq Bioscience | A13094 |
| ALK5 inhibitor II | Selleck | S7223 |
| Liothyronine Sodium (T3) | Selleck | S4217 |
| Isoxazole 9 (ISX9) | Selleck | S7914 |
| γ-Secretase Inhibitor XX | Calbiochem | 565789 |
| (Xxi) | | |
| R428 | Selleck | S2841 |
| N-Acetyl-L-cysteine | Sigma | A9165 |
| (Nac) | | |
| Forskolin (FSK) | Selleck | S2449 |
| ZnSO₄ | Sigma | Z0251 |
| Heparin sodium | Selleck | S1346 |
| MCDB131 | Gibco | 10372-019 |
| DMEM-basic | Gibco | C11965500BT |
| Glucose | Sigma-Aldrich | G7021 |
| GlutaMAX | Gibco | 35050-061 |
| Pen/Strep | Gibco | 15140-122 |
| B27 | Gibco | 12587-010 |
| BSA | Sigma-Aldrich | A4612 |

Cryopreservation and recovery of hPSC-islets.

Cryopreservation and recovery were performed as previously described (Du, Y. et al., 2022, supra).

Cryopreservation. hPSC-islets were dissociated using Accutase and rinsed with DMEM-basic. After counting with a Countess II Automated Cell Counter (Invitrogen, AMQAX1000), cells were cryopreserved at a concentration of 1× 10⁷ cells/mL with a cryopreservation medium consisting of 35% FBS, 5% DMSO (Sigma-Aldrich, D2650), 60% Stage 6 medium and 10 μM Y27632. The vials were then transferred into Thermo Fisher Scientific Mr. Frosty (5100-0001) immediately and frozen in a −80° C. freezer for 24 h. Subsequently, the vials were transferred into liquid nitrogen for long-term storage.

Recovery. Cryopreserved vials were thawed in a 37° C. water bath. Each cell suspension was then transferred into a 15-mL centrifuge tube containing 10 mL of DMEM-basic medium, followed by centrifugation at 350 g for 3 min. Cells were resuspended in DMEM-basic medium supplemented with 1% B27 and 10 μM Y27632. After verifying viability and yield, the cells were seeded at 5×10⁶ cells/well in six-well AggreWell Microwell Plates and spun down at 300 g for 5 min into the microwells. After incubation in 5% CO₂ at 37° C. for 24 h, the clusters were transferred into ultra-low attachment six-well plates containing DMEM-basic supplied with 1% B27. Suspended aggregates were cultured in an incubator shaker at a rotation rate of 90 r.p.m. at 37° C., 5% CO₂ and 85% humidity for 24 h and then used for transplantation.

### Flow Cytometry

Clusters were dissociated into single cells with Accutase for 5-10 min in a 37° C. water bath and then stained for intracellular markers as described previously (Du, Y. et al., 2022, supra). In brief, individual cells were fixed and permeabilized with Fixation/Permeabilization Solution (BD BioSciences, 554714) for 20 min at 4° C. Cells were then washed twice in Perm/Wash buffer (BD BioSciences, 554714) and incubated with primary antibodies overnight at 4° C. in Perm/Wash buffer. Cells were washed three times in Perm/Wash buffer, incubated with secondary antibodies for 1 h at 4° C. in Perm/Wash buffer, and then washed three times in Perm/Wash buffer and analyzed using BD CellQuest Pro. FlowJo version 10 software was used for flow cytometry analysis. The antibodies used are listed in Table 4.

TABLE 4
Antibodies for flow cytometry and immunohistochemistry

| Antigen | Species | Source | Dilution | Cat# |
|---|---|---|---|---|
| PDX1 | Goat | R&D | 1:200 | AF2419 |
| NKX6.1 | Mouse | DSHB | 1:200 | F55A12-c |
| NKX6.1 | Rabbit | Novus | 1:200 | NBP1-49672 |
| NKX2.2 | Mouse | DSHB | 1:200 | 74.5A5-c |
| C-Peptide | Rat | DSHB | 1:200 | GN-ID4 |
| GLUCAGON | Mouse | Sigma-Aldrich | 1:200 | G2654 |
| GLUCAGON | Rabbit | Abcam | 1:500 | Ab92517 |
| MAFA | Rabbit | Novus | 1:200 | NB400-137 |
| UCN3 | Rabbit | The Salk Institute | 1:500 | N/A |
| | | for Biological | | |
| | | Studies | | |
| SOMATOSTATIN | Mouse | Santa Cruz | 1:200 | Sc-55565 |
| CHGA | Rabbit | ZSGB-Bio | 1:500 | ZA-0507 |
| OCT4 | Mouse | BD Biosciences | 1:200 | 611203 |
| NANOG | Rabbit | Abcam | 1:200 | ab21624 |
| CK19 | Mouse | Abcam | 1:1000 | Ab7754 |
| Stem121 | Mouse | Cellartis | 1:1000 | Y40410 |
| CD3 | Rabbit | Servicebio | 1:400 | GB13014 |
| CD20 | Mouse | Servicebio | 1:200 | GB14030 |
| CD68 | Mouse | Servicebio | 1:400 | GB14043 |
| CD31 | Rabbit | Abcam | 1:200 | Ab28364 |

Immunohistochemistry and immunofluorescence staining.

For frozen tissue sections. Clusters or tissue samples were washed with PBS and fixed with 4% PFA for 2 h (hPSC-islet clusters) or 24 h (tissue samples) at 4° C. The samples were washed three times with PBS and dehydrated overnight at 4° C. in 30% sucrose solution. The dehydrated samples were overlaid with OCT (Sakura, 4583), frozen in liquid nitrogen and stored at −80° C. 10-μm cryosections were cut and placed on slides, which were washed with PBS and permeabilized with PBST solution (PBS+0.2% Triton X-100+5% donkey serum) for 1 h at room temperature. The slides were then incubated with primary antibodies diluted in PBST solution at 4° C. overnight. After three washes with PBS, the slides were incubated with secondary antibodies conjugated to Alexa Fluor 488, 555 or 647 (Life Technologies) in PBST solution at 1:1,000 for 1 h and stained with DAPI for 5 min at room temperature. Images were captured using Leica TCS SP8 confocal microscope and Zeiss LM710 confocal microscope.

For paraffin sectioning. Samples were fixed in 10% formalin solution for 7 d at room temperature, paraffin-embedded and sectioned. Sections were deparaffinized, rehydrated and washed in PBS. Hematoxylin and eosin (H&E) staining was performed after rehydration. For immunohistochemistry, slides were submerged in pre-heated antigen retrieval solution and microwaved until boiling for at least 15 min, after which they were left to cool to room temperature. The slides were washed with PBS for 5 min, submerged in blocking reagent for 1 h at room temperature in a humidified, light-protected chamber and washed with PBS for three times. Next, the sections were stained with primary antibodies and secondary antibodies, followed by chromogenic reaction and hematoxylin staining. All antibodies used are listed in Table 4.

Quantitative assessment of the graft volume. The tissue samples of each transplantation site were overlaid with OCT and serially sectioned into 10-μm thick slides using a freezing microtome. Sections were stained with human cell marker Stem121 and imaged using KF-Pro-005 digital slice scanner. The Stem121 positive human grafts were labeled using K-Viewer to measure the total area of grafts. The volume of grafts was calculated by multiplying the total area with the thickness of section.

qRT-PCR

Total RNA was extracted from clusters using the RNeasy Micro Kit (Qiagen, 74004) following the manufacturer's instructions. Transcript One-Step gDNA Removal and cDNA Synthesis SuperMix (TransGen Biotech, AT311-03) was used to synthesize cDNA. KAPA SYBR FAST Universal qPCR Mix (KAPA Biosystems, KK4601) was used for qRT-PCR analysis, which was performed on a 7500 Real Time PCR system. The relative expression levels were normalized to the housekeeping gene GAPDH, and the results were analyzed using ΔΔCt methodology. Primer sequences are listed in Table 5.

TABLE 5
Primers used for qRT-PCR

| | | SEQ | | SEQ |
|---|---|---|---|---|
| Gene | Forward primer sequence | ID No | Reverse primer sequence | ID No |
| GAPDH | TGCACCACCAACTGCTTAGC | 1 | GGCATGGACTGTGGTCATGAG | 2 |
| PDX1 | CGGAACTTTCTATTTAGGAT | 3 | AAGATGTGAAGGTCATACTG | 4 |
| | GTGG | | GCTC | |
| NKX6.1 | GGGCTCGTTTGGCCTATTCG | 5 | CCACTTGGTCCGGCGGTTCT | 6 |
| | TT | | | |
| NKX2.2 | TTCCAGAACCACCGCTACAA | 7 | GGGCGTCACCTCCATACCT | 8 |
| | G | | | |
| MAFA | GCTCTGGAGTTGGCACTTCT | 9 | CTTCAGCAAGGAGGAGGTCA | 10 |
| INS | GCAGCCTTTGTGAACCAACA | 11 | CCCCGCACACTAGGTAGAGA | 12 |
| | C | | | |
| GCG | CATTCACAGGGCACATTCAC | 13 | CGGCCAAGTTCTTCAACAAT | 14 |
| ARX | CTGCTGAAACGCAAACAGA | 15 | CTCGGTCAAGTCCAGCCTCAT | 16 |
| | GGC | | G | |
| FOXA2 | CTGAGCGAGATCTACCAGTG | 17 | CAGTCGTTGAAGGAGAGCGA | 18 |
| | GA | | GT | |
| SST | CGCTGTCCATCGTCCTG | 19 | GGGCATCATTCTCCGTCTG | 20 |
| HHEX | TCAGAATCGACGCGCTAAAT | 21 | AGAGCTATCCAAAGAAGCAC | 22 |
| | G | | CT | |
| NEUROD1 | ATTGCACCAGCCCTTCCTTT | 23 | ACTCGGCGGACGGTTCGTGTT | 24 |
| | GAT | | T | |

### Glucose Stimulated Insulin Secretion (GSIS)

Krebs buffer was prepared by dissolving 129 mM NaCl, 2.5 mM CaCl₂), 4.8 mM KCl, 1.2 mM KH₂PO₄, 1.2 mM MgSO₄, 1 mM Na₂HPO₄, 5 mM NaHCO₃, 10 mM HEPES, and 0.1% BSA in deionized and sterile filtered water. Separate batches of Krebs buffer containing 2.8 mM glucose, 16.7 mM glucose, and 30 mM KCl, respectively, were prepared and warmed to 37° C. hPSC-islets (20-50 clusters) were collected and washed twice with Krebs buffer in a 24-well plate. Cells were then incubated successively in Krebs buffer, Krebs buffer containing 2.8 mM glucose, Krebs buffer containing 16.7 mM glucose and Krebs buffer containing 30 mM KCl at 37° C. for 1 h. Supernatant was collected and cells were washed with fresh Krebs buffer after each incubation. Supernatant samples were frozen at −80° C. for C-peptide testing. After the assay, cells were dispersed into individual cells with Accutase and counted with Countess™ II Automated Cell Counter.

Nonhuman primate transplantation.

All experimental procedures were approved by the Institutional Animal Care and Use Committee of the Institute of Medical Biology, Chinese Academy of Medical Science (Ethics number: DWLL201908013). Seven rhesus macaques (Macaca mulatta) were used for hPSC-islets transplantation. Three healthy monkeys were used to evaluate the feasibility of transplanting hPSC-islets under the anterior rectus sheath, in the subcutaneous space and in the brachioradialis muscle, while four diabetic monkeys were used to validate the functionality of hPSC-islets when transplanted under the anterior rectus sheath.

Diabetes induction. Diabetes was induced with a single intravenously administered dose of STZ (AdooQ, A10868) intravenous injection as described previously (Du, Y. et al., 2022, supra). In brief, STZ (90 mg/kg) was diluted in 0.1 M citrate buffer (pH 4.3-4.5) and immediately injected intravenously (within 5 min) into the overnight fasted macaques. Hydration was followed by administration of saline (50 mL) to each monkey. In order to prevent nausea and vomiting, omeprazole (0.5 mg/kg, Losec®, Astrazeneca AB) was injected intravenously after hydration. Blood glucose was monitored every hour in the first 12 h after STZ injection, after which it was monitored 4 times per day. Exogenous insulin injections commenced 3 d after STZ treatment. The short-acting form of insulin (Humalog®, Eli Lilly Italia S. p. A.) and long-acting form of insulin (Lantus®, Sanofi-Aventis Deutschland GmbH) were injected subcutaneously. Short-acting insulin was administered according to the dosing chart in Table 6. The levels of blood glucose, exogenous insulin, C-peptide, and HbAlc were recorded before hPSC-islet transplantation.

TABLE 6
Dose adjustments chart for short-acting
insulin, based on blood glucose levels.

| Blood glucose (mM) | Insulin dosing (IU) |
|---|---|
| <3 | 0 |
| ≤5 | 0.5 |
| ≤10 | 1 |
| ≤15 | 1.5 |
| ≤20 | 2 |
| ≤25 | 2.5 |
| ≤30 | 3 |
| >30 | 3.5 |

Immunosuppression. The immunosuppression regimen was started 9 days before transplantation (day 0) as previously described (Du, Y. et al., 2022, supra). Detailed information are listed in Table 7. Induction therapy: rituximab was injected on day −9. ATG was infused on days −5 and −3. Basiliximab was given on days 0 and 2 post-transplantation. Methylprednisolone, chlorphenamine maleate tablets and diphenhydramine were administered 10 minutes before rituximab and ATG treatments to reduce allergic reactions. Maintenance therapy: belatacept was administered on days 0, 4 and 14, after which it was injected biweekly; sirolimus and tacrolimus were administered daily. The dosages of sirolimus and tacrolimus were adjusted according to trough blood levels (tacrolimus: 4-10 ng/ml; sirolimus: 4-10 ng/mL). Blood concentrations of the drugs were tested using Viva-E (Vital Scientific N.V.).

TABLE 7
Drugs used for immunosuppression and preemptive treatment.

| | Administration | |
|---|---|---|
| Drugs | method | Manufacturer |
| Rituximab | 375 mg/m², i.v. | Rituxan ®, La Roche Ltd |
| ATG | 5 mg/kg, i.v. | Thymoglobuline ®, Genzyme |
| | | Polyclonals S.A.S |
| Basiliximab | 0.3 mg/kg, i.v. | Simulect ®, Novartis Pharma |
| | | Schweiz AG |
| Methylprednisolone | 10 mg/kg, i.v. | Solu-Medrol ®, Pfizer |
| | | Manufacturing Belgium NV |
| Chlorphenamine | 0.1 mg/kg, p.o. | Chengdu First Pharmaceutical |
| Maleate Tablets | | Co., Ltd |
| Diphenhydramine | 0.5 mg/kg, p.o. | Linfen baozhu Pharmaceutical |
| | | Co., Ltd |
| Belatacept | 20 mg/kg, i.v. | Nulojix ®, Bristol-Myers Squibb |
| Sirolimus | q.d., p.o. | Rapamune ®, Pfizer Ireland |
| | | Pharmaceuticals |
| Tacrolimus | b.i.d., p.o. | Prograf ®, Astellas Ireland Co., |
| | | Ltd |
| CVF | 100 U/kg, i.v. | Quidel |
| Etanercept | 5 mg/kg, i.v. or | Enbrel ®, Pfizer Ireland |
| | 2.5 mg/kg, i.h. | Pharmaceuticals |
| Valganciclovir | 20 mg/kg, p.o. | Valcyte ®, La Roche Ltd |

Prophylactic and preemptive treatment. To prevent CMV infection, valganciclovir was administered daily from day −4 onwards. To alleviate inflammatory reactions, cobra venom factor (CVF) was administered on day −1 and etanercept was administered on day 0 (5 mg/kg, i.v.), as well as on days 3, 7 and 10 (2.5 mg/kg, i.h.) post-transplantation.

Anesthesia. Anesthesia was initiated with propofol (0.5 mL/kg, Petsun Therapeutics) and maintained with isoflurane and oxygen. Heart rate, temperature, blood oxygenation and blood pressure were monitored in real time during the hPSC-islets transplantation. The overall quality of hPSC-islet preparations is described in FIG. 1 and Table 8 and 9.

Subcutaneous transplantation of hPSC-islets. hPSC-islets (1×10⁴ IEQ) were dispersed in IVM as reported (Yu, M. et al. Islet transplantation in the subcutaneous space achieves long-term euglycaemia in preclinical models of type 1 diabetes. Nat Metab 2, 1013-1020, doi: 10.1038/s42255-020-0269-7 (2020)) and loaded into syringe with a puncture needle (0.7× 80 TWLB). After sterile preparation of the scalp, the syringe containing the hPSC-islets was inserted into the subcutaneous space under the guidance of ultrasound. The total volume of inoculum was slowly infused into the subcutaneous space and the needle point was then swabbed with iodophor.

Brachioradialis muscle hPSC-islets transplantation. hPSC-islets (1×10⁴ IEQ) were dispersed in saline as reported (Bertuzzi, F., Colussi, G., Lauterio, A. & De Carlis, L. Intramuscular islet allotransplantation in type 1 diabetes mellitus. European review for medical and pharmacological sciences 22, 1731-1736, doi: 10.26355/eurrev_201803_14588 (2018)) and loaded into syringe with a puncture needle (0.7×80 TWLB). After sterile preparation of the forearm, the syringe containing the hPSC-islets was inserted distally in the fiber direction of the brachioradialis muscle and mobilized through the muscle to the proximal part with the guidance of ultrasound. The total volume of inoculum was infused into the space between the fibers of the brachioradialis muscle while slowly moving the needle from proximal to distal part of the muscle to obtain a pearls-on-a-string distribution of hPSC-islets in the muscle and the needle point was then swabbed with iodophor.

Sub-anterior rectus sheath hPSC-islets transplantation. hPSC-islets were dispersed in saline and then loaded into syringe with a puncture needle (0.7×80 TWLB). With the guidance of ultrasound imaging, the hPSC-islet suspension was injected into the space between the anterior rectus sheath and rectus abdominis. For each monkey, 8 injections were applied with 4 injections on each side. In detail, a 10 MHz ultrasonic probe was used, which was placed around the navel to show the short axial section of the rectus abdominis muscle. When transplanted under the left side of the anterior rectus sheath, the puncture needle entered from the confluence of the external oblique, internal oblique and transversus abdominia aponeurosis. When transplanted under the right side of the anterior rectus sheath, the puncture needle entered from the right edge of the left abdomen lineaalba. When the needle tip pierced the anterior rectus sheath and reached the edge of the rectus abdominis, the puncture needle was inserted into the space between the anterior rectus sheath and rectus abdominis, with the puncture needle very close to the anterior layer of the rectus sheath. After reaching the target position, hPSC-islets were injected while the puncture needle was withdrawn. As a result, hPSC-islets were dispersed in and around the needle track.

Routine tests. C-peptide secretion, HbA1c, complete blood count, serum creatinine and liver function analysis were routinely performed. A complete blood cell count was performed using a Sysmex XT-200i. HbA1c, serum creatinine and liver function analysis were assessed using a Mindray BS-2000.

Intravenous glucose tolerance test (IVGTT). After an overnight fast, 0.75 g/kg body weight of 50% glucose was infused intravenously into the monkey within 1 min. Blood samples were collected at 0, 5, 15, 30, 60, and 90 min post-injection. Blood glucose was measured with a handheld glucometer, and C-peptide levels were measured by ELISA.

Arginine stimulation test. C-peptide secretion responses to intravenous arginine stimulation were measured at two different plasma glucose levels (Ref). In brief, after an overnight fast, a dose of 70 mg/kg of 10% arginine hydrochloride (Sigma, Cat #A5006) was administered over 30 s, the beginning of which was designated 0 min. Blood samples were collected at 0, 2, 4 and 10 min after the first pulse of arginine. A 50% glucose was then injected to raise and further maintain the plasma glucose levels at 20 mM. Fifty minutes after the first pulse of arginine, a second arginine pulse (70 mg/kg) was injected. The blood samples were collected at 0, 2, 4 and 10 min after the second pulse of arginine. The C-peptide levels were measured by ELISA.

### ELISA

C-peptide was detected using a human C-peptide ELISA kit (ALPCO, 80-CPTHU-E10) according to the manufacturer's instructions. ELISA was performed with three technical replicates for all samples.

### Autopsy and Histological Analysis

Full necropsy of all monkeys was performed by an experienced primate pathologist. Tissue specimens of the major organs were fixed in 4% PFA and 10% formalin for cryosections and paraffin sections, respectively.

### Statistical Analysis.

Data analysis was performed using GraphPad Prism software. Statistical significance was evaluated by t-test. Throughout the manuscript, n represents number of biological replicates unless otherwise stated. P-values are presented as follows: *P<0.05; **P<0.005; ***P<0.0005; ****P<0.00005.

### Example 1. Comparison of Sub-Rectus Sheath Transplantation of Islets with Two Otherextraperitoneal Transplantation Strategies

To evaluate the feasibility of transplanting hPSC-islets into the rectus sheath, sub-rectus sheath strategy was first compared with two previously reported extraperitoneal islet transplantation strategies: intramuscular transplantation and the subcutaneous transplantation (FIG. 1a) (Yu, M. et al. 2020, supra; Bertuzzi, F. et al., 2020, supra; Rafael, E. et al. Intramuscular autotransplantation of pancreatic islets in a 7-year-old child: a 2-year follow-up. Am J Transplant 8, 458-462, doi: 10.1111/j. 1600-6143.2007.02060. x (2008); Sakata, N. et al. Strategy for clinical setting in intramuscular and subcutaneous islet transplantation. Diabetes Metab Res Rev 30, 1-10, doi: 10.1002/dmrr. 2463 (2014)).

FIG. 1a illustrates the three transplantation sites. In this example, the methods used for transplantation in the brachioradialis muscle and in subcutaneous space were based on previous reports (Yu, M. et al. 2020, supra; Bertuzzi, F. et al., 2020, supra). For transplantation under the rectus sheath, the space between the anterior rectus sheath and rectus abdominis was chosen as a graft site to distance from the peritoneum and to avoid the latent surgical injury to the vascular and neural networks surrounding the posterior rectus sheath (FIG. 1b) (Sevensma, K. E., Leavitt, L. & Pihl, K. D. Anatomy, Abdomen and Pelvis, Rectus Sheath. StatPearls (book) (2022)).

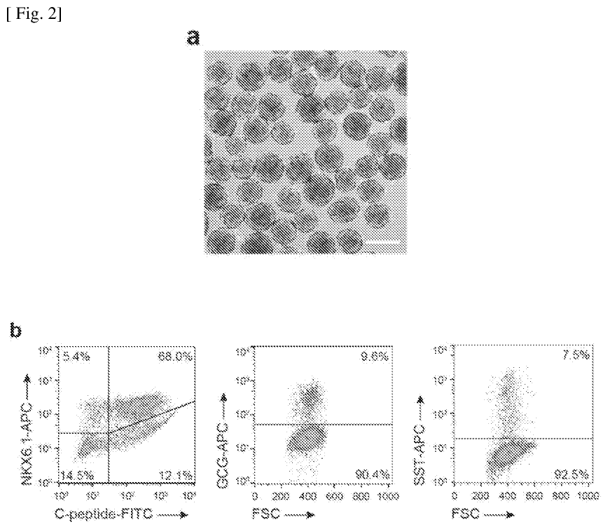

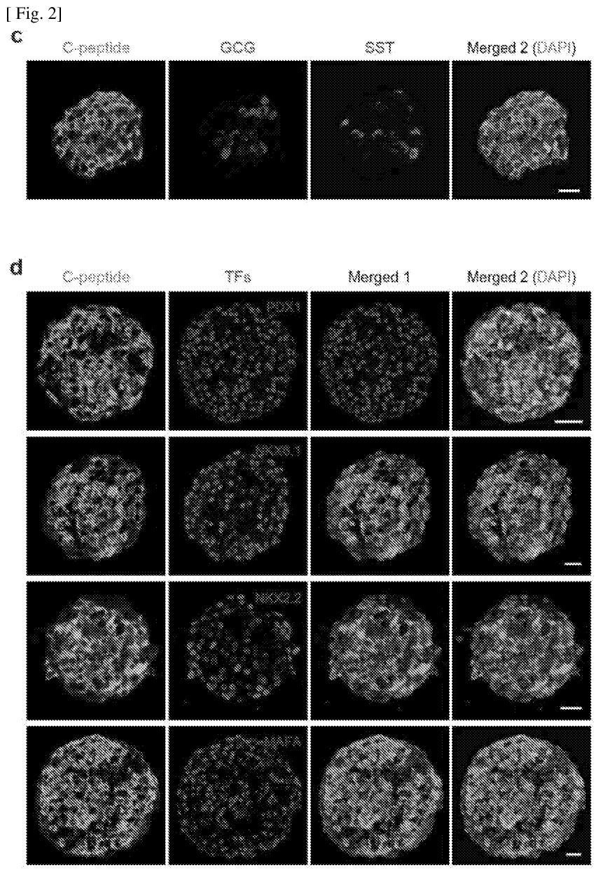

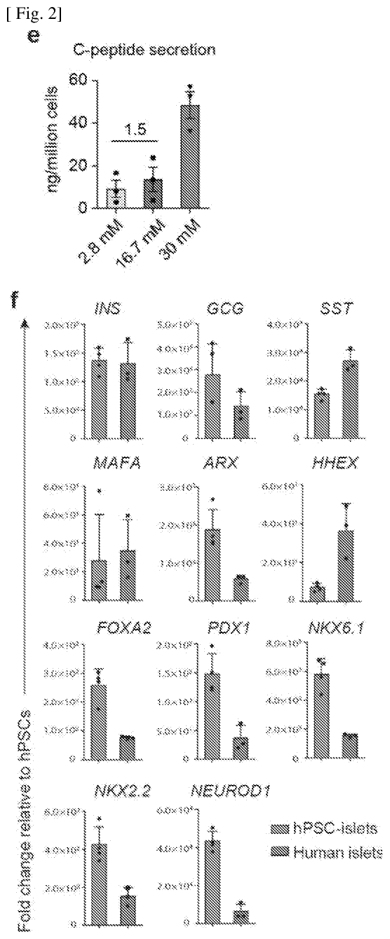

Three healthy adult rhesus macaques (Macaca mulatta) (Monkeys 1-3) were used (Table 8). hPSC-islets were differentiated from hPSCs and cryopreserved in single-cell suspensions as described in the section entitled “Methods” as above. Two days before transplantation, hPSC-islets were recovered and reaggregated, the characterization of which was shown in FIG. 2. Immunosuppressive therapy was applied as described in the section entitled “Methods” as above. Under the guidance of ultrasound, hPSC-islets were transplanted into into each monkey at three selected at a dose of approximately 8× 10⁴ islet equivalent (IEQ) per site (FIG. 1a-b and Table 8).

TABLE 8
Characterizations of rhesus macaque recipients and their respective transplanted hPSC-islets for transplantation site test.

| | | | | hPSC-islets | | | | | | | |
|---|---|---|---|---|---|---|---|---|---|---|---|
| Recipient | | | | | Transplanted | Transplanted | | | Flow cytometry | | |
| | | | Body | | cell | cell | Recovery | | C-peptide⁺ | | |
| | | Age | Weight | | Number/site | Dose/site | Viability | Yield | NKX6.1⁻ | GCG+ | SST+ |
| ID | Gender | (years) | (kg) | Cell line | (*10⁸) | (IEQ) | (%) | (%) | (%) | (%) | (%) |
| 1# | F | 5 | 4.5 | hPSC-#8 | 1.96 | 78954 | 85.00 | 80.50 | 66.7 | 8.3 | 7.2 |
| 2# | M | 5 | 7.1 | hPSC-#8 | 2.01 | 80765 | 83.00 | 88.73 | 67.5 | 7.9 | 5.8 |
| 3# | M | 5 | 8.1 | hPSC-#8 | 2.08 | 83662 | 87.25 | 82.50 | 70.3 | 8.1 | 12.0 |

TABLE 9
Characterizations of diabetic rhesus macaque recipients and
their respective transplanted hPSC-islets for sub-anterior rectus sheath transplantation.

| | | | | | hPSC-islets | | |
|---|---|---|---|---|---|---|---|
| Recipients | | | | | Transplanted | Transplanted | Transplanted |
| | | | Body | | cell | cell | cell dose/ |
| | | Age | Weight | | number | dose | needle tracts |
| ID | Gender | (years) | (kg) | Cell line | (#10⁸) | (IEQ/kg) | (IEQ/tract) |
| 4# | M | 6 | 6.4 | hPSC-#8 | 6.44 | 40542 | 32434 |
| 5# | F | 5 | 5.7 | hPSC-#8 | 6.76 | 47594 | 33911 |
| 6# | M | 5 | 7.8 | hPSC-#8 | 9.35 | 47991 | 46791 |
| 7# | M | 5 | 7.3 | hPSC-#8 | 7.93 | 43907 | 40065 |

| | | | | | hPSC-islets | | | | | | | | | |
|---|---|---|---|---|---|---|---|---|---|---|---|---|---|---|
| Recipients | | | | | | | Flow cytometry | | | | | | | |
| | | | | | Recovery | | C- | | | | | | | |
| | | | Body | | Via- | | peptide⁺ | | | | | | | |
| | Gen- | Age | Weight | Cell | bility | Yield | NKX6.1⁺ | GCG+ | SST+ | qRT-PCR (Relative to S0) | | | | |
| ID | der | (years) | (kg) | line | (%) | (%) | (%) | (%) | (%) | OCT4 | NANOG | INS | GCG | SST |
| 4# | M | 6 | 6.4 | hPSC- | 87.25 | 82.50 | 70.3 | 8.1 | 12.0 | 3.05E−04 | 1.90E−02 | 7.59E+03 | 7.73E+04 | 1.72E+03 |
| | | | | #8 | | | | | | | | | | |
| 5# | F | 5 | 5.7 | hPSC- | 86.00 | 80.50 | 63.3 | 6.4 | 9.3 | 1.36E−04 | 5.84E−02 | 3.12E+03 | 4.57E+04 | 1.12E+03 |
| | | | | #8 | | | | | | | | | | |
| 6# | M | 5 | 7.8 | hPSC- | 86.00 | 80.50 | 63.3 | 6.4 | 9.3 | 1.36E−04 | 5.84E−02 | 3.12E+03 | 4.57E+04 | 1.12E+03 |
| | | | | #8 | | | | | | | | | | |
| 7# | M | 5 | 7.3 | hPSC- | 82.00 | 90.00 | 76.5 | 7.6 | 5.4 | 1.05E−04 | 2.17E−02 | 1.51E+04 | 1.26E+05 | 7# |
| | | | | #8 | | | | | | | | | | |

### Cell Survival at Early Stage Post-Transplantation

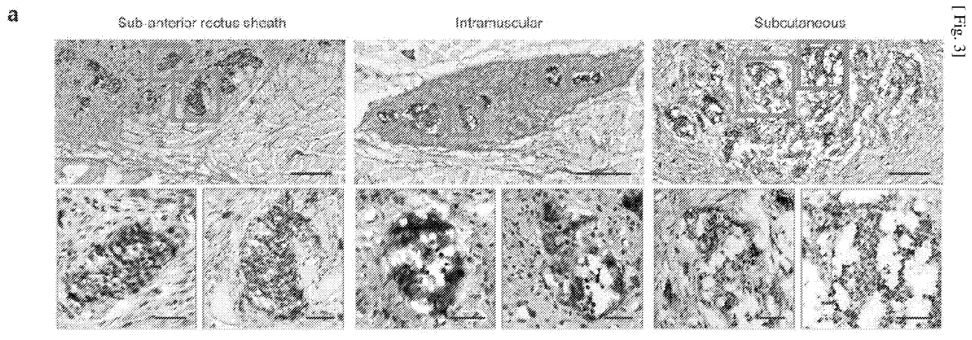

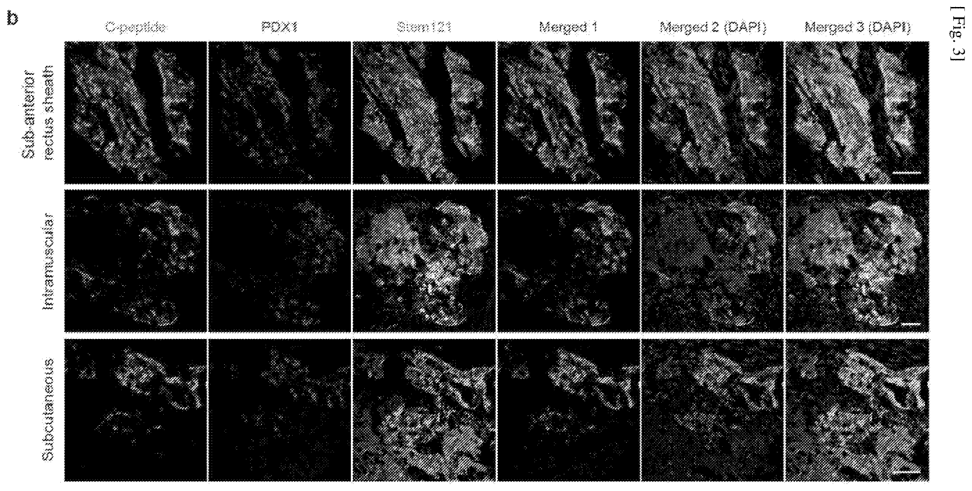

Monkey 1 was sacrificed at 1 week post transplantation (wpt) to assess the early stage cell survival and gene expression pattern of hPSC-islet grafts. By immunostaining of the human cell marker Stem121, hPSC-islet grafts in all three sites were detected (FIG. 3a).

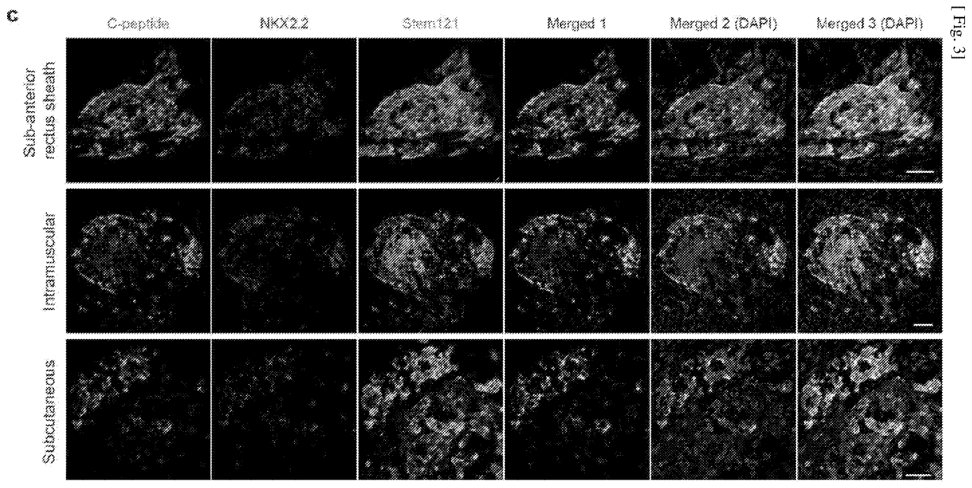

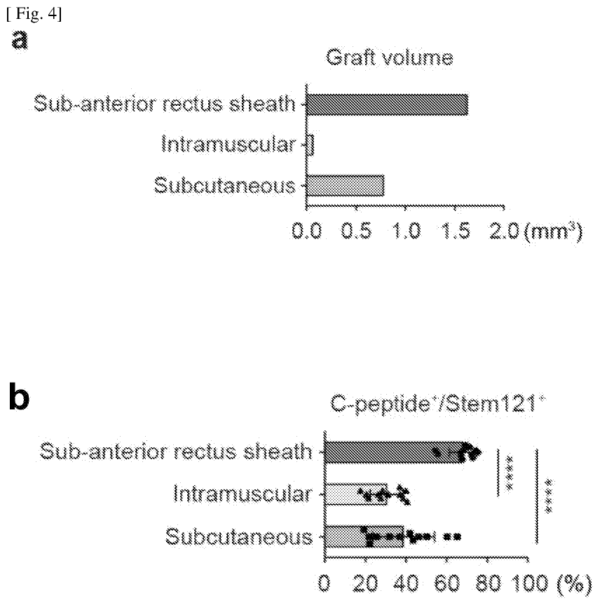

As shown in FIG. 4a, the volumes of human grafts in the brachioradialis muscle and subcutaneous space were each dramatically less than that that under the anterior rectus sheath. Furthermore, the proportion of C-peptide positive β cells was significantly lower in intramuscular grafts and in subcutaneous grafts, which also showed reduced expression of pancreatic transcription factors (NKX6.1, PDX1 and NKX2.2), as compared to the proportion or expression level of the transplants before transplantation (FIG. 4b-d and FIG. 3b-c). In contrast, hPSC-islet grafts under the anterior rectus sheath maintained structural integrity with robust expression of islet hormones and transcription factors (FIG. 4c-d and FIG. 3b-c).

Collectively, these results suggest that hPSC-islets showed a better early stage survival and maintenance when transplanted under the anterior rectus sheath.

### Cell Survival at Later Stage Post-Transplantation

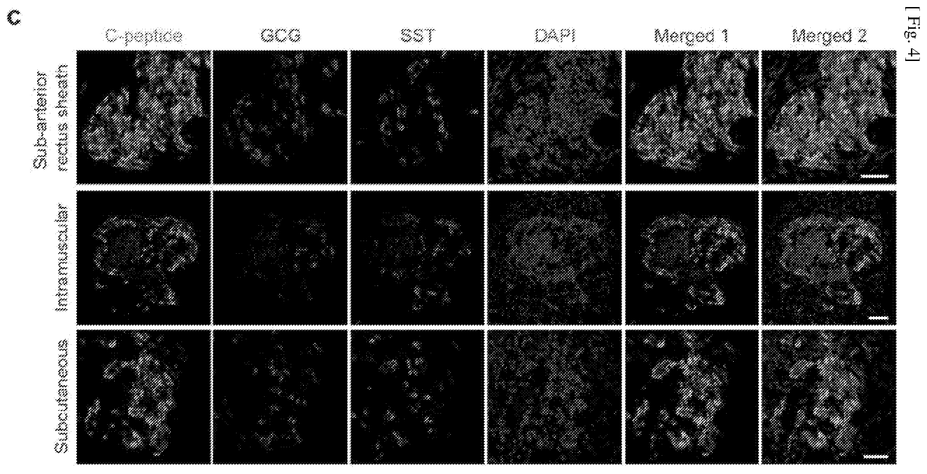

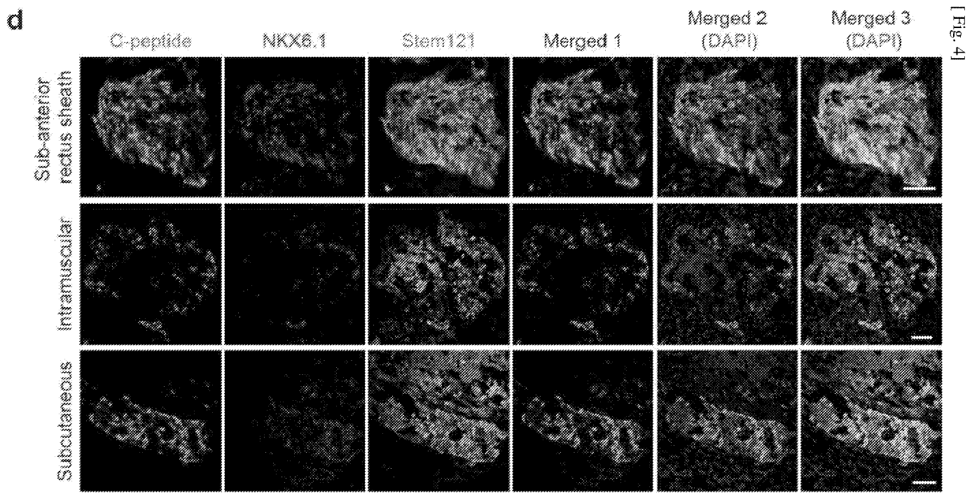

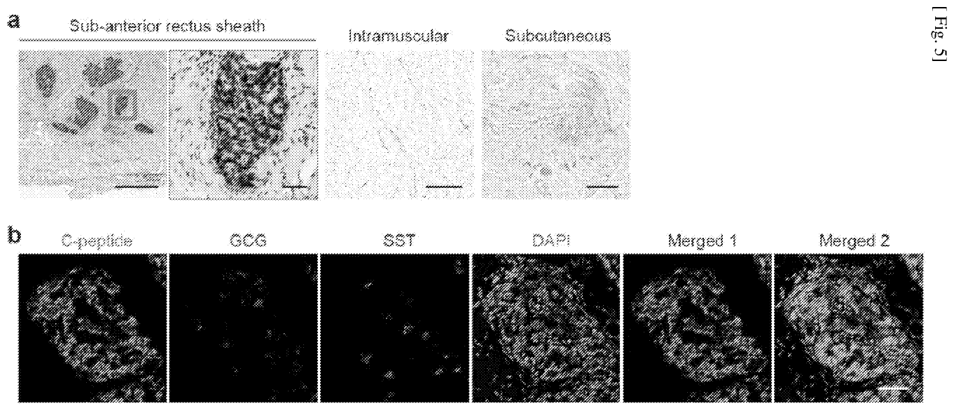

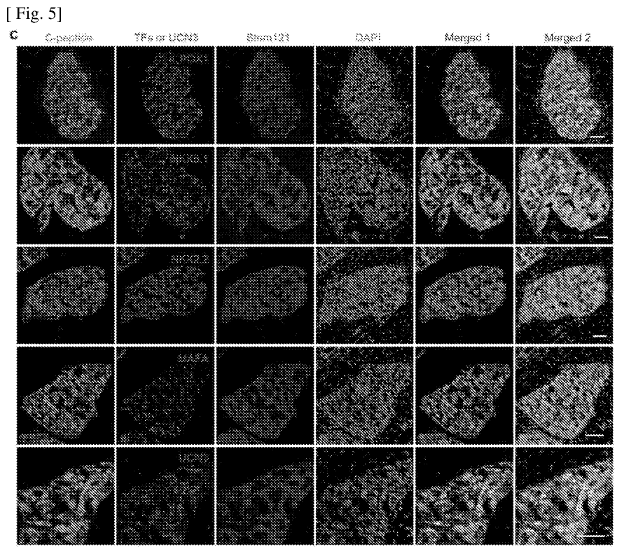

Monkey 2 and Monkey 3 were sacrificed at 4 wpt. By serial sectioning of the 4-week grafts and staining of Stem121, it was observed that human cells could only be detected in grafts grown under the anterior rectus sheath, while hPSC-islets injected into the brachioradialis muscle and subcutaneous space were completely eliminated. The findings from both monkeys were consistent (FIG. 5a and FIG. 6a).

Notably, it was found that most of the hPSC-islets were grafted between the surface of the rectus abdominis and the anterior rectus sheath, while a small proportion was seeded into the rectus abdominis (FIG. 5a and FIG. 6a).

### Immunofluorescence Staining of 4-Week Human Grafts Under the Anterior Rectus Sheath

Further analysis of the 4-week human grafts under the anterior rectus sheath was conducted by immunofluorescence staining. The results of immunofluorescence staining showed that the human grafts expressed essential islet hormones and transcription factors (FIG. 5b-c and FIG. 6b-c). Notably, mature β cell markers MAFA and UCN3 were detectable (FIG. 5c and FIG. 6c) (Blum, B. et al. Functional beta-cell maturation is marked by an increased glucose threshold and by expression of urocortin 3. Nat Biotechnol 30, 261-264, doi: 10.1038/nbt. 2141 (2012); Kaneto, H. et al. PDX-1 and MafA play a crucial role in pancreatic beta-cell differentiation and maintenance of mature beta-cell function. Endocrine journal 55, 235-252, doi: 10.1507/endocrj. k07e-041 (2008)). In addition, abundant blood vessels infiltrated into human grafts was observed (FIG. 5d and FIG. 6d). Immunopathological analysis showed the presence of rare CD3+ T cells, CD20+ B cells and CD68+ macrophages within or surrounding the grafts (FIG. 5e and FIG. 6e). Collectively, these results suggested that the sub-anterior rectus sheath provided an environment facilitating functional maturation and maintenance of hPSC-islets.

### Example 2. Functionality Research of Sub-Aterior Rectus Sheath Transplanted hPSC-Islets in Diabetic Monkeys

In this example, the function of hPSC-islets in ameliorating diabetic conditions when transplanted under anterior rectus sheath in diabetic monkeys was investigated.

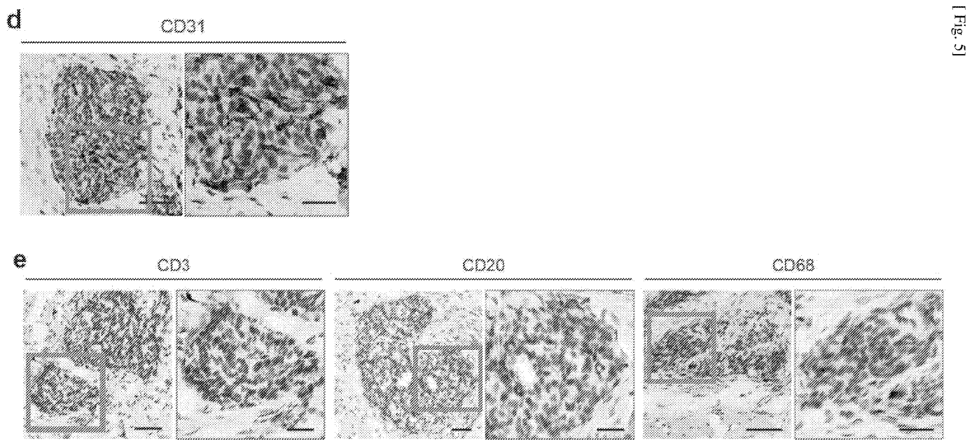

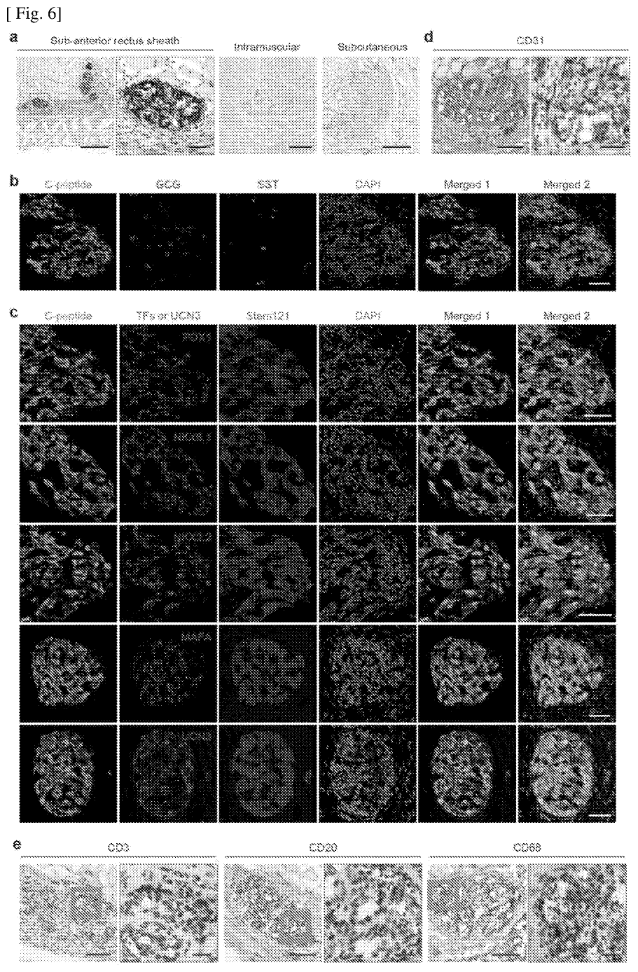

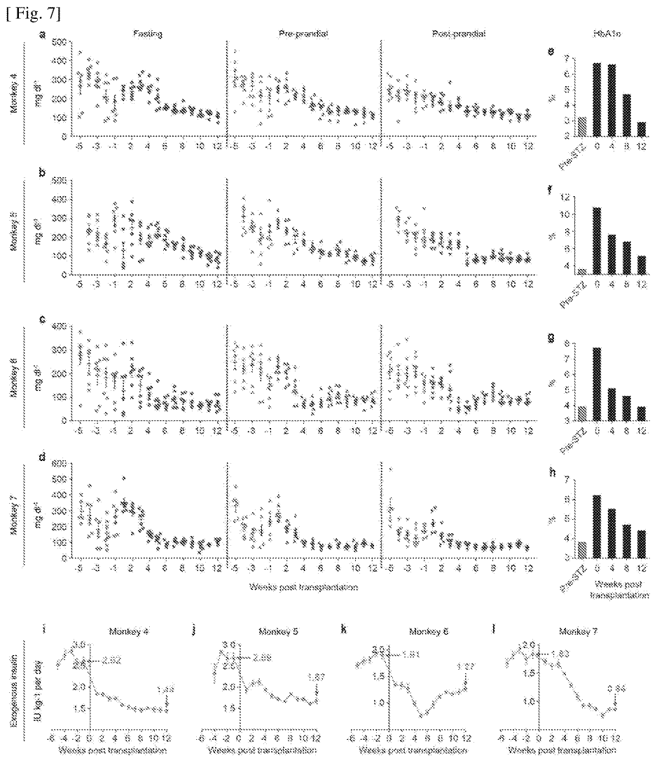

Four adult monkeys (Monkeys 4-7) were injected with a single high-dose streptozotocin (STZ) to induce a diabetic steate as reported (Table 9) (Du, Y. et al., 2022, supra; Zhu, H., Yu, L., He, Y. & Wang, B. Nonhuman primate models of type 1 diabetes mellitus for islet transplantation. J Diabetes Res 2014, 785948, doi: 10.1155/2014/785948 (2014)). After STZ injection, all macaques showed significantly increased blood glucose levels and extremely low C-peptide concentrations (fasting C-peptide: 0.08±0.07 ng/ml; post-prandial C-peptide: 0.08±0.07 ng/ml) (FIGS. 7a-d, 8a-d and 9a-d). Exogenous insulin was administered 3 days after STZ-injection according to blood glucose levels, the daily dose of which ranged from 1.5 to 3.0 IU kg⁻¹ per day, consistent with previous reports in the same model system (FIG. 7i-1) (Du, Y. et al., 2022, supra; Zhu, H., et al., 2014, supra; Shin, J. S. et al. Long-term control of diabetes in immunosuppressed nonhuman primates (NHP) by the transplantation of adult porcine islets. Am J Transplant 15, 2837-2850, doi: 10.1111/ajt. 13345 (2015)). HbAlc, a parameter commonly used to evaluate long-term average blood glucose concentration (Sherwani, S. I., Khan, H. A., Ekhzaimy, A., Masood, A. & Sakharkar, M. K. Significance of HbAlc Test in Diagnosis and Prognosis of Diabetic Patients. Biomark Insights 11, 95-104, doi: 10.4137/BMI. S38440 (2016); American Diabetes, A. 2. Classification and Diagnosis of Diabetes: Standards of Medical Care in Diabetes-2021. Diabetes Care 44, S15-S33, doi: 10.2337/dc21-S002 (2021)), was dramatically elevated from 3.6±0.3% before STZ treatment to 7.7±2.2% on the day of hPSC-islet transplantation (FIG. 7e-h).

The dose of hPSC-islets for the sub-anterior rectus sheath transplantation was designed based on that used in intraportal infusion, ranging from 40,000 to 48,000 IEQ per kilogram of body weight (IEQ/kg) and averaging at 45,000 IEG/kg (Table 9) (Du, Y. et al., 2022, supra). hPSC-islets were injected into the space between the anterior rectus sheath and rectus abdominis using a puncture needle in a tubular manner, and cell clusters were dispersed in and around the needle track (FIG. 1b). Considering that a high density of hPSC-islets in a single needle track could impair cell survival, hPSC-islets were administered by 8 injections with 4 needle tracks in parallel on each side of the rectus abdominis (FIG. 1c). Diabetic recipients were studied by continuously monitoring blood glucose and C-peptide secretion during 12 weeks post transplantation to assess the durability of the functional effects of hPSC-islets, given that xeno-immune attack mediated clearance of hPSC-islet grafts was observed after 4 mpt under human-to-monkey xeno-transplantation setting as previously reported (Du, Y. et al., 2022, supra).

### Improvements in Glycemic Control

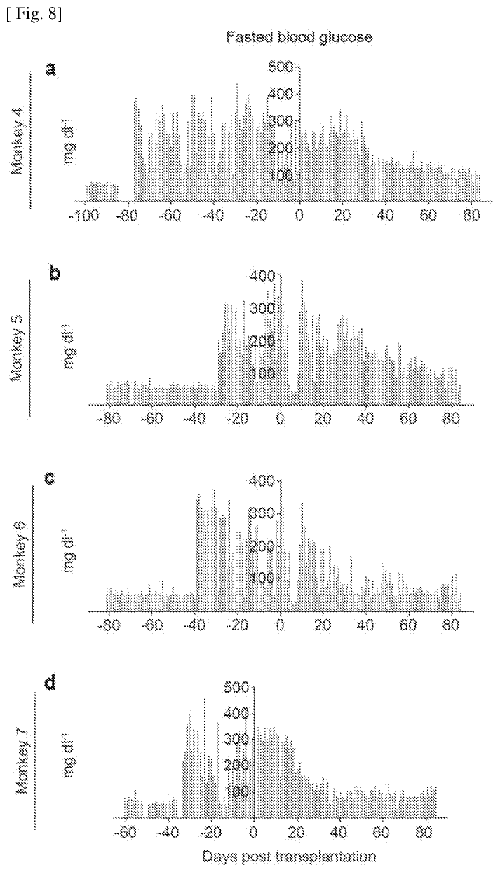

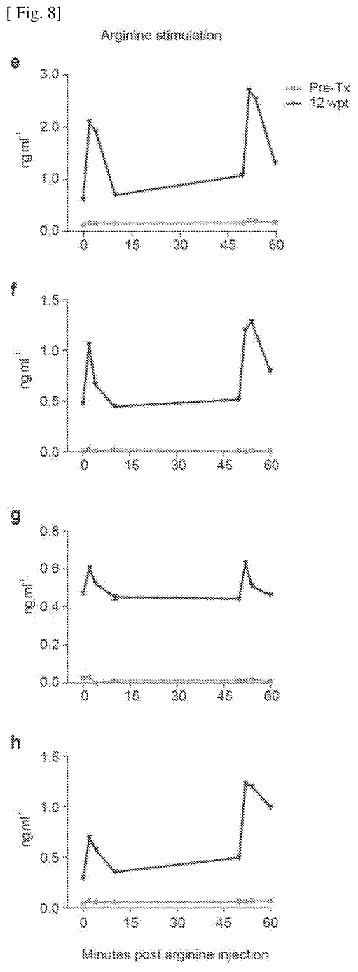

After injection of hPSC-islets into the sub-anterior rectus sheath, all diabetic recipients showed a significant improvement in overall glycemic control (FIG. 7). Levels of blood glucose were gradually decreased, and this change was accompanied by a reduction of exogenous insulin requirements (FIGS. 7a-d and 7i-1). Fasting blood glucose was stably maintained under 180 mg dl⁻¹ (10 mM) from 6 wpt and blood glucose two hours post-meal fell to a level below 144 mg dl⁻¹ (8.0 mM) from 5 wpt in all recipients (FIG. 7a-d and FIG. 8a-d). Fluctuation of blood glucose levels was also greatly attenuated (FIG. 7a-d). Consequently, the average Hb1Ac went from 7.7±2.2% at baseline to 4.3±1.3% at 12 wpt (FIG. 7e-h).

Notably, severe hypoglycemic events, which occurred in the perioperative period after intraportal infusion, did not detected in recipient macaques after sub-anterior rectus sheath transplantation. In a previous study of the present inventors, when hPSC-islets were infused into the hepatic portal vein, instant blood-mediated inflammatory reaction (IBMIR)-mediated β cell death frequently resulted in blood glucose levels below 54 mg dl⁻¹ (3.0 mM) in the recipient macaques in the first 12 hours after transplantation, and intravenous injections of high-concentration dextrose were required (Du, Y. et al., 2022, supra; Rickels, M. R. & Robertson, R. P. Pancreatic Islet Transplantation in Humans: Recent Progress and Future Directions. Endocr Rev 40, 631-668, doi: 10.1210/er. 2018-00154 (2019); Shin, J. S. et al., 2015, supra; Faradji, R. N. et al. C-peptide and glucose values in the peritransplant period after intraportal islet infusions in type 1 diabetes. Transplant Proc 37, 3433-3434, doi: 10.1016/j. transproceed. 2005.09.090 (2005)). These distinct observations suggested the improved early-stage survival of hPSC-islets following sub-anterior rectus sheath transplantation.

### Reduced Exogenous Insulin Requirement

The exogenous insulin requirement was reduced in all recipients of sub-anterior rectus sheath hPSC-islet transplantation (FIG. 7i-1). A sharp reduction in the exogenous insulin requirement was observed immediately after hPSC-islet injection, which was followed by a continued gradual decrease, trends that correspond with functional maturation of hPSC-islets (FIG. 7i-1). At 12 wpt, the average exogenous insulin requirement decreased from 2.3±0.5 IU kg⁻¹ per day to 1.3±0.4 IU kg⁻¹ per day (FIG. 7i-1).

### C-Peptide Secretion

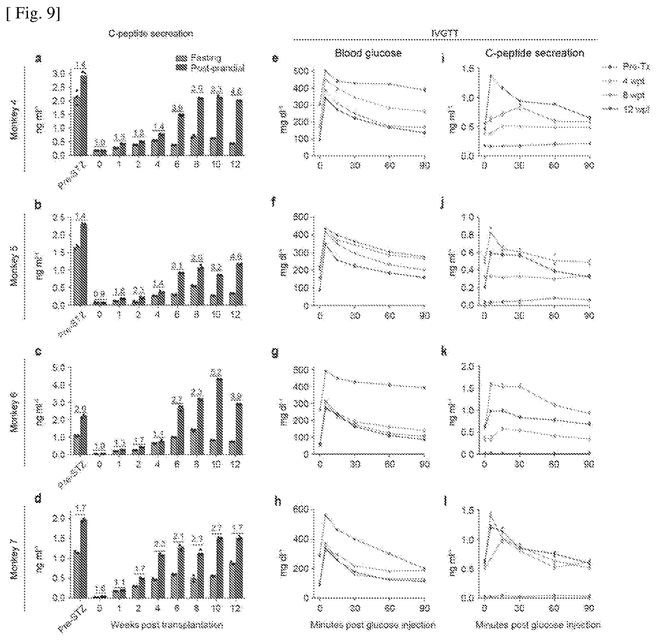

The C-peptide secretion in all monkeys was also monitored. Both fasting C-peptide and postprandial C-peptide significantly increased in the first 6 wpt, with average levels of approximately 0.6 ng ml⁻¹ and 2.0 ng ml⁻¹, respectively, maintained from 8 wpt (FIG. 9a-d). The stimulation indices after 6 wpt were comparable with those of native islets (FIG. 9a-d).

In addition, the intravenous glucose tolerance test (IVGTT) showed that the glucose clearance capacity was gradually improved along with the enhancement of glucose-responsive insulin secretion (FIG. 9e-1).

Besides, arginine-stimulated insulin secretion test provided further evidence of the C-peptide secretion capacity of hPSC-islet grafts (FIG. 8e-h).

Collectively, these data revealed that by restoration of insulin secretion, hPSC-islets transplanted under the anterior rectus sheath effectively improved the overall glycemic control of diabetic macaques.

### Histological Analysis

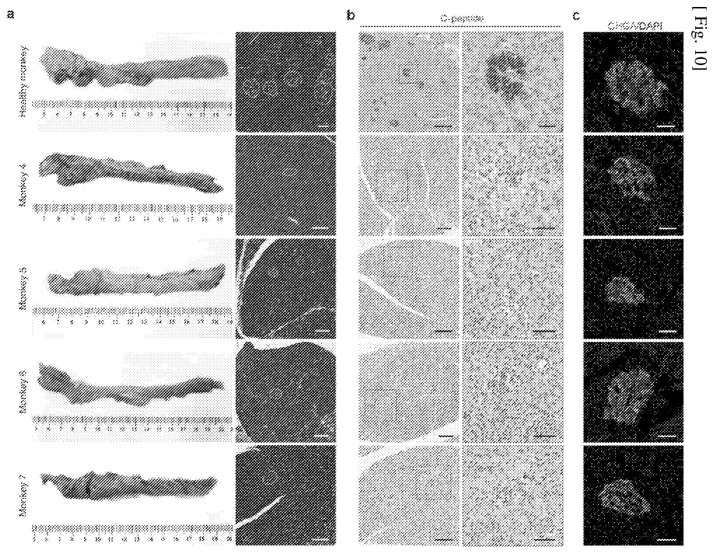

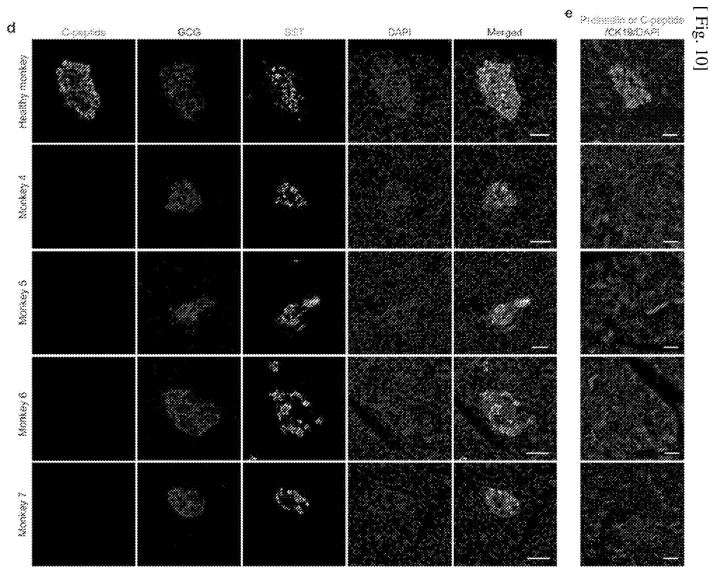

To confirm that the improved blood glucose levels resulted from hPSC-islet transplantation rather than endogenous β cell recovery, all recipient monkeys were sacrificed at 13 wpt for evaluation. Histological analysis of native pancreata revealed that the endogenous islets were severely destroyed and C-peptide⁺ cells were undetectable (FIG. 10a-d). These results were consistent with the extremely low concentrations of endogenous C-peptide that were observed before transplantation (FIGS. 9a-d and 9 i-1). Furthermore, the CK19⁺Proinsulin⁺ cells were not observed, which population has been reported to contribute to endogenous β cell recovery in the pancreas after STZ treatment (FIG. 10e) (Bottino, R. et al. Recovery of endogenous beta-cell function in nonhuman primates after chemical diabetes induction and islet transplantation. Diabetes 58, 442-447, doi: 10.2337/db08-1127 (2009)).

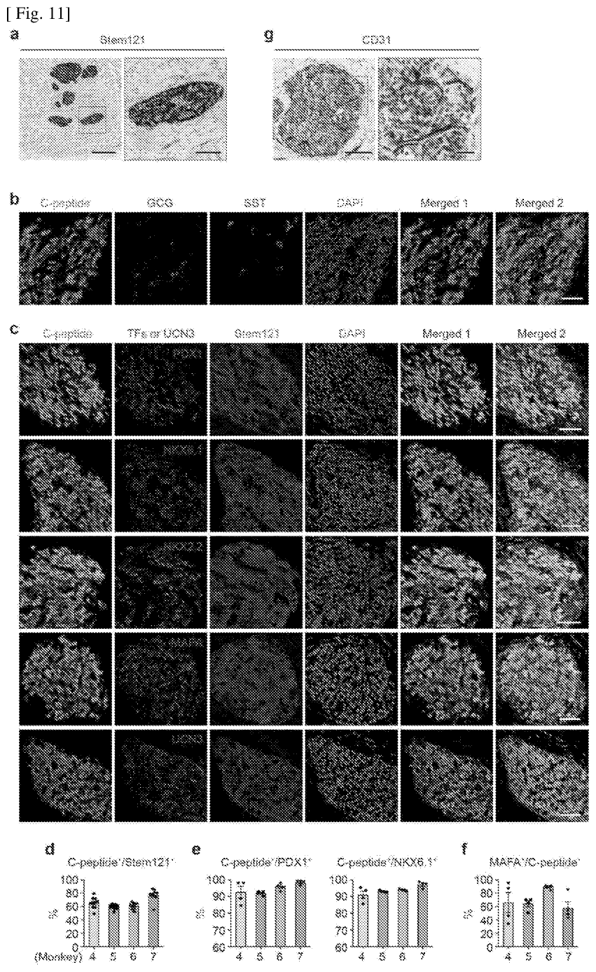

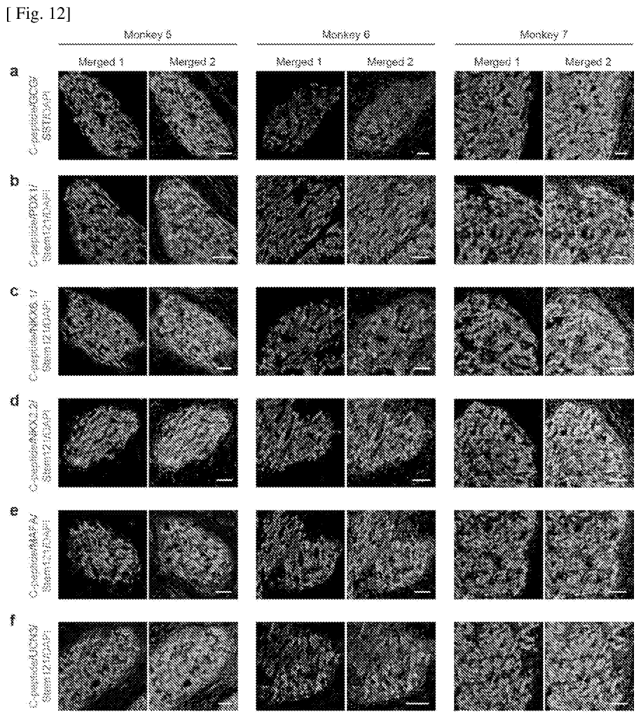

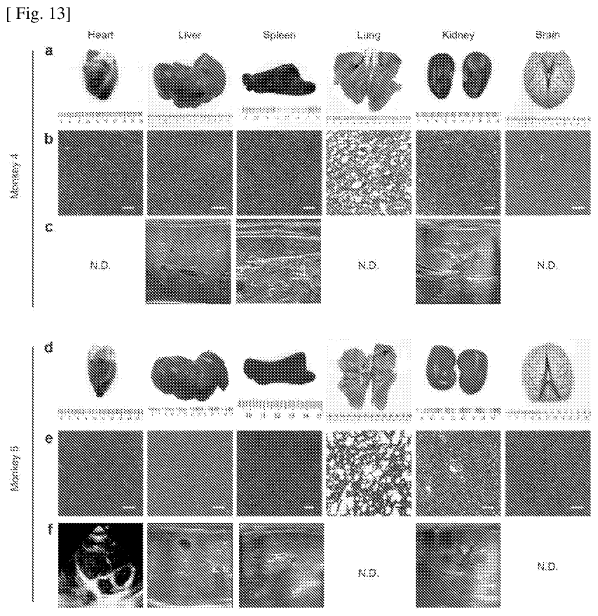

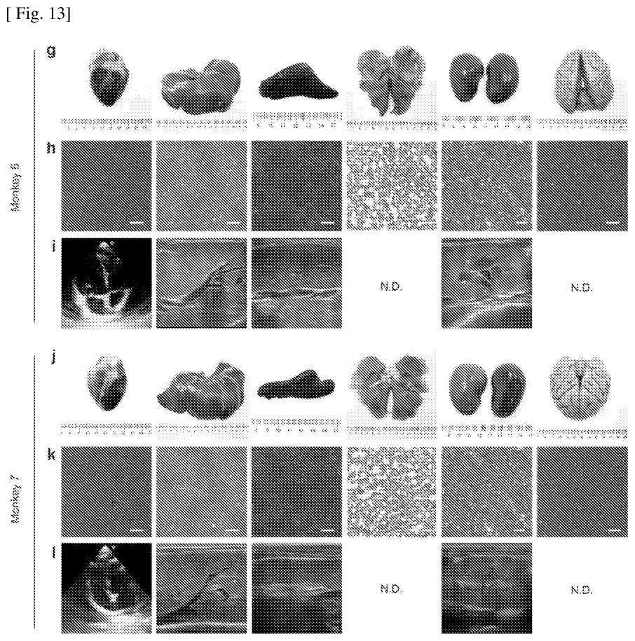

In contrast, high proportion of C-peptide⁺ β cells were present in the human grafts under the anterior rectus sheath, while robust expression of MAFA and UCN3 was detected (FIG. 11c). Quantitative analysis showed that the proportion of β cells in the engrafted hPSC-islets was similar to that measured before transplantation, and more than half of the β cells co-stained with MAFA (FIG. 11d-f). These observations were consistent in the four diabetic monkeys (FIG. 11d-f and FIG. 12a-f).

Collectively, these results showed evidence attributing the improved glycemic control to hPSC-islet grafts under the anterior rectus sheath.

### Systemic Ultrasonography

In addition, systemic ultrasonography was carried out on all four recipient monkeys at 13 wpt before sacrifice and no evidence of teratoma formation was found (FIG. 12). Full autopsies further confirmed the absence of tumorigenesis and abnormality in the major organs being examined (FIG. 12).

1. A method for transplanting one or more cells, or one or more tissues to a subject in need thereof, comprising introducing the cells or tissues at a site under the rectus sheath.

2. The method of claim 1, the cells or tissues are introduced at a site between the rectus sheath and the musculus rectus abdominis.

3. A method for transplanting one or more cells, or one or more tissues to a subject in need thereof, wherein the method allows one or more of the transplanted cells or tissues to graft at a site under the rectus sheath after transplantation.

4. The method of claim 3, wherein the method allows one or more of the transplanted cells or tissues to graft at a site between the rectus sheath and the musculus rectus abdominis.

5. The method of claim 3 or 4, wherein one or more of the transplanted cells graft into musculus rectus abdominis.

6. The method of any one of claims 1 to 5, wherein the rectus sheath is anterior rectus sheath or posterior rectus sheath, preferably anterior rectus sheath.

7. The method of any one of claims 1 to 6, wherein the cells or tissues are cells or tissues generated in vitro or isolated from native organ.

8. The method of claim 7, wherein the cells or tissues are generated in vitro from one or more of pluripotent cells, multipotent cells, progenitor cells or precursor cells.

9. The method of claim 8, wherein the cells or tissues are cells or tissues differentiated from human pluripotent stem cells (hPSCs).

10. The method of claim 9, wherein the cells are hormone-secreting cells differentiated from hPSCs.

11. The method of claim 10, wherein the cells are islets differentiated from hPSCs (hPSC-islets).

12. The method of any one of claims 9 to 11, wherein the hPSCs are embryonic stem cells (ESCs) or induced pluripotent stem cells (iPSCs).

13. The method of any one of claims 8 to 12, wherein at least a portion of the transplanted cells or tissues undergo maturation after the transplantation.

14. The method of any one of claims 1 to 13, wherein the cells are introduced at the site by injection.

15. A method for treating a disease or condition caused by or related to insufficiency of insulin in a subject in need thereof, comprising transplanting one or more islets to the subject, wherein the one or more islets are introduced at a site under the rectus sheath.

16. A method for reducing the requirement of exogenous insulin of a subject having a disease or condition caused by or related to insufficiency of insulin, comprising transplanting one or more islets to the subject, wherein the one or more islets are introduced at a site under the rectus sheath.

17. The method of claim 15 or claim 16, wherein the disease is diabetes, or a complication thereof.

18. The method of any one of claims 15 to 17, wherein the islets are introduced at a site between the rectus sheath and the musculus rectus abdominis.

19. The method of any one of claims 15 to 18, wherein the method allows one or more of the transplanted islets to graft at a site under the rectus sheath after transplantation.

20. The method of claim 19, wherein the method allows one or more of the transplanted islets to graft at a site between the rectus sheath and musculus rectus abdominis.

21. The method of claim 19 or claim 20, wherein one or more of the transplanted islets graft into musculus rectus abdominis.

22. The method of any one of claims 15 to 21, wherein the rectus sheath is anterior rectus sheath or posterior rectus sheath, preferably anterior rectus sheath.

23. The method of any one of claims 15-22, wherein the islets are generated in vitro or isolated from pancreas.

24. The method of any one of claims 15-23, wherein the islets are generated in vitro from one or more of pluripotent cells, multipotent cells, progenitor cells or precursor cells derived from hPSCs.

25. The method of claim 24, wherein the islets are differentiated from hPSCs (hPSC-islets).

26. The method of claim 25, wherein the hPSCs are ESCs or iPSCs.

27. The method of any one of claims 15 to 26, wherein at least a portion of the transplanted islets undergo maturation after the transplantation.

28. The method of any one of claims 15 to 27, wherein the cells are introduced at the site by injection.

29. The method of any one of claims 15-28, wherein the islets are transplanted in an amount of 100 to 200,000 islet equivalent (IEQ).

30. The method of any one of claims 15-29, wherein the islets are transplanted at both the left and right rectus abdominis at a site under the corresponding rectus sheath.

31. The method of any one of claims 15-30, comprising transplanting islets to the subjects for more than one time.

32. Use of hPSC-islets for treating a disease or condition caused by or related to insufficiency of insulin, wherein the hPSC-islets are transplanted at a site under the anterior rectus sheath.

33. Use of hPSC-islets for reducing the requirement of exogenous insulin, wherein the hPSC-islets are transplanted at a site under the anterior rectus sheath.

34. The use of claim 32 or claim 33, wherein the hPSC-islets are transplanted at a site between the anterior rectus sheath and the musculus rectus abdominis.

35. A device for injecting islets to a site within the rectus sheath, and preferably between the rectus sheath and the musculus rectus abdominis.

36. The device of claim 35, wherein the islets are hPSC-derived islets.

37. The device of claim 35 or claim 36, wherein the device comprises a needle.

38. The device of claim 35 or claim 36, wherein the device comprises multiple needle tracks.

39. The device of claim 38, wherein the device comprises 2, 3, 4, 5, 6, 7, 8, 9, 10 or more needle tracks.

40. The device of any one of claims 35 to 39, wherein the device comprising a cartridge.

41. The device of claim 40, wherein the cartridge comprises islets in an amount of 100 to 200,000 islet equivalent (IEQ) per kg body weight of the recipient.

42. The device of claim 40, wherein the cartridge comprises islets in an amount of 1×? 10² to 2×10⁷ IEQ.


SEQUENCE LISTING (24 sequences)

SEQ ID NO: 1 - DNA, 20 bp, synthetic construct
  misc_feature 1..20: note = Primer
  source 1..20: mol_type = other DNA
tgcaccaccaactgcttagc

SEQ ID NO: 2 - DNA, 21 bp, synthetic construct
  misc_feature 1..21: note = Primer
  source 1..21: mol_type = other DNA
ggcatggactgtggtcatgag

SEQ ID NO: 3 - DNA, 24 bp, synthetic construct
  misc_feature 1..24: note = Primer
  source 1..24: mol_type = other DNA
cggaactttctatttaggatgtgg

SEQ ID NO: 4 - DNA, 24 bp, synthetic construct
  misc_feature 1..24: note = Primer
  source 1..24: mol_type = other DNA
aagatgtgaaggtcatactggctc

SEQ ID NO: 5 - DNA, 22 bp, synthetic construct
  misc_feature 1..22: note = Primer
  source 1..22: mol_type = other DNA
gggctcgtttggcctattcgtt

SEQ ID NO: 6 - DNA, 20 bp, synthetic construct
  misc_feature 1..20: note = Primer
  source 1..20: mol_type = other DNA
ccacttggtccggcggttct

SEQ ID NO: 7 - DNA, 21 bp, synthetic construct
  misc_feature 1..21: note = Primer
  source 1..21: mol_type = other DNA
ttccagaaccaccgctacaag

SEQ ID NO: 8 - DNA, 19 bp, synthetic construct
  misc_feature 1..19: note = Primer
  source 1..19: mol_type = other DNA
gggcgtcacctccatacct

SEQ ID NO: 9 - DNA, 20 bp, synthetic construct
  misc_feature 1..20: note = Primer
  source 1..20: mol_type = other DNA
gctctggagttggcacttct

SEQ ID NO: 10 - DNA, 20 bp, synthetic construct
  misc_feature 1..20: note = Primer
  source 1..20: mol_type = other DNA
cttcagcaaggaggaggtca

SEQ ID NO: 11 - DNA, 21 bp, synthetic construct
  misc_feature 1..21: note = Primer
  source 1..21: mol_type = other DNA
gcagcctttgtgaaccaacac

SEQ ID NO: 12 - DNA, 20 bp, synthetic construct
  misc_feature 1..20: note = Primer
  source 1..20: mol_type = other DNA
ccccgcacactaggtagaga

SEQ ID NO: 13 - DNA, 20 bp, synthetic construct
  misc_feature 1..20: note = Primer
  source 1..20: mol_type = other DNA
cattcacagggcacattcac

SEQ ID NO: 14 - DNA, 20 bp, synthetic construct
  misc_feature 1..20: note = Primer
  source 1..20: mol_type = other DNA
cggccaagttcttcaacaat

SEQ ID NO: 15 - DNA, 22 bp, synthetic construct
  misc_feature 1..22: note = Primer
  source 1..22: mol_type = other DNA
ctgctgaaacgcaaacagaggc

SEQ ID NO: 16 - DNA, 22 bp, synthetic construct
  misc_feature 1..22: note = Primer
  source 1..22: mol_type = other DNA
ctcggtcaagtccagcctcatg

SEQ ID NO: 17 - DNA, 22 bp, synthetic construct
  misc_feature 1..22: note = Primer
  source 1..22: mol_type = other DNA
ctgagcgagatctaccagtgga

SEQ ID NO: 18 - DNA, 22 bp, synthetic construct
  misc_feature 1..22: note = Primer
  source 1..22: mol_type = other DNA
cagtcgttgaaggagagcgagt

SEQ ID NO: 19 - DNA, 17 bp, synthetic construct
  misc_feature 1..17: note = Primer
  source 1..17: mol_type = other DNA
cgctgtccatcgtcctg

SEQ ID NO: 20 - DNA, 19 bp, synthetic construct
  misc_feature 1..19: note = Primer
  source 1..19: mol_type = other DNA
gggcatcattctccgtctg

SEQ ID NO: 21 - DNA, 21 bp, synthetic construct
  misc_feature 1..21: note = Primer
  source 1..21: mol_type = other DNA
tcagaatcgacgcgctaaatg

SEQ ID NO: 22 - DNA, 22 bp, synthetic construct
  misc_feature 1..22: note = Primer
  source 1..22: mol_type = other DNA
agagctatccaaagaagcacct

SEQ ID NO: 23 - DNA, 23 bp, synthetic construct
  misc_feature 1..23: note = Primer
  source 1..23: mol_type = other DNA
attgcaccagcccttcctttgat

SEQ ID NO: 24 - DNA, 22 bp, synthetic construct
  misc_feature 1..22: note = Primer
  source 1..22: mol_type = other DNA
actcggcggacggttcgtgttt